(19) United States
(12) Patent Application Publication
(10) Pub. No.: US 2021/0137995 A1
(43) Pub. Date: May 13, 2021

(54) MATERIAL DEPOSITING SYSTEM FOR TREATING A PATIENT

(71) Applicant: Fractyl Laboratories, Inc., Lexington, MA (US)

(72) Inventors: Harith Rajagopalan, Wellesley Hills, MA (US); David Maggs, Boston, MA (US); Jay Caplan, Boston, MA (US); Thomas C. Kochem, Watertown, MA (US); J. Christopher Flaherty, Auburndale, FL (US)

(73) Assignee: Fractyl Laboratories, Inc., Lexington, MA (US)

(21) Appl. No.: 16/905,274

(22) Filed: Jun. 18, 2020

Related U.S. Application Data
(63) Continuation of application No. PCT/US19/12338, filed on Jan. 4, 2019.
(60) Provisional application No. 62/613,888, filed on Jan. 5, 2018.
(60) Provisional application No. 62/739,445, filed on Oct. 1, 2018.

(51) Int. Cl. A61K 35/38; A61B 10/02; A61B 18/08

(52) U.S. Cl. CPC A61K 35/38; A61B 2018/00577; A61B 18/08; A61B 10/02

(57) ABSTRACT

A system for treating a medical condition comprises: a harvesting device for harvesting tissue from a mammalian subject at a harvest site; and a depositing device for depositing material in a patient at a deposit site, the material based on the harvested tissue. The deposited material is configured to generate resultant tissue configured to treat the medical condition of the patient. Methods are also provided for treating a medical condition of a patient.

### CROSS-REFERENCE TO RELATED APPLICATIONS

This application is a continuation of PCT Application No. PCT/US2019/012338 (Attorney Docket no. 41714-717.301), filed Jan. 4, 2019, which claims the benefit of U.S. Provisional Application Ser. No. 62/613,888 (Attorney Docket No. 41714-717.101, Client Docket No. MCT-036-PR1), entitled “Material Depositing System for Treating a Patient”, filed Jan. 5, 2018, and U.S. Provisional Application Ser. No. 62/739,445 (Attorney Docket No. 41714-717.102, Client Docket No. MCT-036-PR2), entitled “Material Depositing System for Treating a Patient”, filed Oct. 1, 2018, the content of each of which is incorporated herein by reference in its entirety for all purposes.

This application is related to: U.S. patent application Ser. No. 13/945,138 (Attorney Docket No. 41714-703.301, Client Docket No. MCT-001-US), entitled “Devices and Methods for the Treatment of Tissue”, filed Jul. 18, 2013; U.S. patent application Ser. No. 14/470,503 (Attorney Docket No. 41714-704.301, Client Docket No. MCT-002-US), entitled “Heat Ablation Systems, Devices and Methods for the Treatment of Tissue”, filed Aug. 27, 2014; U.S. patent application Ser. No. 14/515,324 (Attorney Docket No. 41714-705.301, Client Docket No. MCT-003-US), entitled “Tissue Expansion Devices, Systems and Methods”, filed Oct. 15, 2014; U.S. patent application Ser. No. 14/609,332 (Attorney Docket No. 41714-706.301, Client Docket No. MCT-004-US), entitled “Electrical Energy Ablation Systems, Devices and Methods for the Treatment of Tissue”, filed Jan. 29, 2015; U.S. patent application Ser. No. 14/609,334 (Attorney Docket No. 41714-707.301, Client Docket No. MCT-005-US), entitled “Ablation Systems, Devices and Methods for the Treatment of Tissue”, filed Jan. 29, 2015; U.S. patent application Ser. No. 14/673,565 (Attorney Docket No. 41714-708.301, Client Docket No. MCT-009-US), entitled “Methods, Systems and Devices for Performing Multiple Treatments on a Patient”, filed Mar. 30, 2015; U.S. patent application Ser. No. 14/956,710 (Attorney Docket No. 41714-709.301, Client Docket No. MCT-013-US), entitled “Methods, Systems and Devices for Reducing the Luminal Surface Area of the Gastrointestinal Tract”, filed Dec. 2, 2015; U.S. patent application Ser. No. 14/917,243 (Attorney Docket No. 41714-710.301, Client Docket No. MCT-023-US), entitled “Systems, Methods and Devices for Treatment of Target Tissue”, filed Mar. 7, 2016; U.S. patent application Ser. No. 15/156,585 (Attorney Docket No. 41714-711.301, Client Docket No. MCT-024-US), entitled “Systems, Devices and Methods for the Creation of a Therapeutic Restriction in the Gastrointestinal Tract”, filed May 17, 2016; U.S. patent application Ser. No. 15/274,948 (Attorney Docket No. 41714-712.301, Client Docket No. MCT-027-US), entitled “Injectate Delivery Devices, Systems and Methods”, filed Sep. 23, 2016; U.S. patent application Ser. No. 15/274,764 (Attorney Docket No. 41714-714.501, Client Docket No. MCT-028-US-CIP1), entitled “Systems, Devices and Methods for Performing Medical Procedures in the Intestine”, filed Sep. 23, 2016; U.S. patent application Ser. No. 15/274,809 (Attorney Docket No. 41714-714.502, Client Docket No. MCT-028-US-CIP2), entitled “Systems, Devices and Methods for Performing Medical Procedures in the Intestine”, filed Sep. 23, 2016; U.S. patent application Ser. No. 15/406,572 (Attorney Docket No. 41714-713.301, Client Docket No. MCT-029-US), filed Jan. 13, 2017; U.S. Provisional Patent Application Ser. No. 62/533,569 (Attorney Docket No. 41714-715.101, Client Docket No. MCT-025-PR1), entitled “Intestinal Catheter Device and System”, filed Jul. 17, 2017; U.S. patent application Ser. No. 15/683,713 (Attorney Docket No. 41714-714.301, Client Docket No. MCT-028-US-CIP1-CON1), entitled “Systems, Devices, and Methods for Performing Medical Procedures in the Intestine”, filed Aug. 22, 2017; International Patent Application Serial Number PCT/US2017/061074 (Attorney Docket No. 41714-716.601, Client Docket No. MCT-035-PCT), entitled “Systems, Devices, and Methods for Performing Medical Procedures in the Intestine”, filed Nov. 10, 2017; U.S. patent application Ser. No. 15/812,969 (Attorney Docket No. 41714-714.302, Client Docket No. MCT-028-US-CIP2-CON1), entitled “Systems, Devices, and Methods for Performing Medical Procedures in the Intestine”, filed Nov. 14, 2017; U.S. Provisional Patent Application Ser. No. 62/739,470 (Attorney Docket No. 41714-718.101, Client Docket No. MCT-037-PR1), entitled “Systems and Methods for Depositing Material in a Patient”, filed Oct. 1, 2018; the contents of each of which is incorporated herein by reference in its entirety for all purposes.

### BACKGROUND OF THE INVENTION

### 1. Field of the Invention

The present invention relates generally to systems for depositing material on and/or in a patient, in particular to systems that harvest tissue, and deposit a material that includes the harvested tissue, in a portion of the patient's gastrointestinal tract.

Bariatric surgeries, such as the Roux-en-Y gastric bypass, have proven their effectiveness in the prevention, treatment, and reversal of a spectrum of cardiovascular, metabolic, and cancer disorders, among others. At least some portion of the metabolic improvement is driven by changes in the hormonal or neuro-hormonal signaling from the intestinal mucosa to the rest of the body, altering insulin production and insulin sensitivity in many organs throughout the body, altering hunger versus satiety, and influencing metabolism in other ways as well. However, bariatric surgeries are invasive, expensive, and carry an important risk of complications. These features limit the scalable use of bariatric surgery to all patients with relevant diseases. There is a need for systems, devices, and methods that deliver a similar metabolic benefit to the surgeries described above but avoid their risks, significant costs, and other limitations.

Disorders of the intestine, such as celiac disease and inflammatory bowel disease, have significant morbidity and mortality for patients around the world. Intestinal procedures in these patients are currently limited to diagnostic procedures, such as mucosal biopsy, or palliative interventional procedures. There is a need for systems, devices, and methods for improved treatment of disorders of the intestine.

### BRIEF SUMMARY OF THE INVENTION

According to an aspect of the present inventive concepts, a system for treating a medical condition, comprising: a harvesting device for harvesting tissue from a mammalian subject at a harvest site; and a depositing device for depositing material in a patient at a deposit site, the material based on the harvested tissue. The deposited material is configured to generate resultant tissue configured to treat the medical condition of the patient.

In some embodiments, the medical condition treated comprises two or more medical conditions of the patient.

In some embodiments, the medical condition treated comprises a medical condition selected from the group consisting of: Type 2 diabetes; Type 1 diabetes; Double diabetes; gestational diabetes; hyperglycemia; pre-diabetes; monogenic diabetes; maturity onset diabetes of the young; impaired glucose tolerance; insulin resistance; hyperinsulinemia; hypoinsulinemia; non-diabetic hypoglycemia; elevated albuminuria; non-alcoholic fatty liver disease; non-alcoholic steatohepatitis; obesity; obesity-related disorder; polycystic ovarian syndrome; hypertriglyceridemia; hypercholesterolemia; psoriasis; gastroesophageal reflux disease; coronary artery disease; stroke; transient ischemic attack; cognitive decline; dementia; Alzheimer's Disease; neuropathy; diabetic nephropathy; retinopathy; heart disease; diabetic heart disease; heart failure; diabetic heart failure; hirsutism; hyperandrogenism; fertility issues; menstrual dysfunction; cancer; liver cancer; ovarian cancer; breast cancer; endometrial cancer; cholangiocarcinoma; adenocarcinoma; glandular tissue tumor; stomach cancer; colorectal cancer; prostate cancer; diastolic dysfunction; hypertension; myocardial infarction; microvascular disease related to diabetes; anorexia nervosa; anorexia; a binge eating disorder; a hyperphagic state; hyperphagia; polyphagia; Prader Willi syndrome; an obesity-related genetic disorder; hypoglycemia; post-bariatric hypoglycemia; recurrent obesity post-bariatric surgery; recurrent metabolic disease post-bariatric surgery; iron overload conditions such as hemochromatosis types 1-4 and/or bantu siderosis; hypercholesterolemia; pancreatic cancer; short bowel syndrome; sleep apnea; arthritis; rheumatoid arthritis; and combinations thereof.

In some embodiments, the medical condition treated comprises at least two medical conditions selected from the group consisting of: Type 2 diabetes; Type 1 diabetes; Double diabetes; gestational diabetes; hyperglycemia; pre-diabetes; monogenic diabetes; maturity onset diabetes of the young; impaired glucose tolerance; insulin resistance; hyperinsulinemia; hypoinsulinemia; non-diabetic hypoglycemia; elevated albuminuria; non-alcoholic fatty liver disease; non-alcoholic steatohepatitis; obesity; obesity-related disorder; polycystic ovarian syndrome; hypertriglyceridemia; hypercholesterolemia; psoriasis; gastroesophageal reflux disease; coronary artery disease; stroke; transient ischemic attack; cognitive decline; dementia; Alzheimer's Disease; neuropathy; diabetic nephropathy; retinopathy; heart disease; diabetic heart disease; heart failure; diabetic heart failure; hirsutism; hyperandrogenism; fertility issues; menstrual dysfunction; cancer; liver cancer; ovarian cancer; breast cancer; endometrial cancer; cholangiocarcinoma; adenocarcinoma; glandular tissue tumor; stomach cancer; colorectal cancer; prostate cancer; diastolic dysfunction; hypertension; myocardial infarction; microvascular disease related to diabetes; anorexia nervosa; anorexia; a binge eating disorder; a hyperphagic state; hyperphagia; polyphagia; Prader Willi syndrome; an obesity-related genetic disorder; hypoglycemia; post-bariatric hypoglycemia; recurrent obesity post-bariatric surgery; recurrent metabolic disease post-bariatric surgery; iron overload conditions such as hemochromatosis types 1-4 and/or bantu siderosis; hypercholesterolemia; pancreatic cancer; short bowel syndrome; sleep apnea; arthritis; rheumatoid arthritis; and combinations thereof.

In some embodiments, the resultant tissue is configured to provide a modified barrier function, and the modified barrier function comprises a barrier function that is different than the barrier function provided by tissue present at the deposit site prior to the depositing of the material. The modified barrier function can be configured to provide a different passage of nutrients and/or a different passage of gut microbiota.

In some embodiments, the medical condition treated comprises diarrhea. The depositing of the material can be configured to increase absorption of: water; electrolyte solutions; and/or other fluids, in the small intestine and/or the large intestine.

In some embodiments, the depositing of the material is configured to modify iron absorption of the patient. The iron absorption can be reduced to treat an iron overload disorder.

In some embodiments, the depositing of the material is configured to modify absorption of fat and/or cholesterol. The depositing of the material can be configured to reduce absorptions in the distal small intestine. The depositing of the material can be configured to reduce absorptions to treat hypercholesterolemia and/or another lipid disorder.

In some embodiments, the depositing of the material is configured to modify Vitamin B12 absorption of the patient. The modification of the Vitamin B12 absorption can comprise an increase in absorption configured to treat pernicious anemia.

In some embodiments, the depositing of the material is configured to treat chronic gastrointestinal wounds of the patient. The material can comprise stem cells and the deposit site can comprise locations proximate the gastrointestinal wounds.

In some embodiments, the depositing of the material is configured to modify an immune reaction of the patient. The depositing of the material can be configured to produce and/or secrete a substance that binds to an antigen known to trigger an autoimmune response in the patient. The substance can comprise an antibody, RNA aptamer, and/or an enzyme. The antigen can trigger gluten sensitivity and/or a gluten allergy. The depositing of the material can be configured to treat a disorder selected from the group consisting of: gluten allergy; gluten sensitivity; Celiac disease; and combinations thereof.

In some embodiments, the harvest site comprises one or more anatomical locations of the patient.

In some embodiments, the harvest site comprises one or more anatomical locations of a mammal that is not the patient.

In some embodiments, the harvest site comprises one or more locations in the ileum and/or colon. The deposit site can comprise one or more locations in the duodenum and/or proximal jejunum. The system can be configured to treat a medical condition selected from the group consisting of: Type 2 diabetes; Type 1 diabetes; insulin resistance; obesity; NAFLD; NASH; PCOS; a binge eating disorder; a hyperphagic state; hyperphagia; Prader Willi syndrome; an obesity-related genetic disorder; and combinations of one, two, or more of these.

In some embodiments, the harvest site comprises one or more locations in the duodenum and/or proximal jejunum. The deposit site can comprise one or more locations in the duodenum and/or the proximal jejunum, and the material can include transgenically modified harvested tissue. The harvested tissue can be transgenically modified to cause resultant tissue. The system can be configured to treat a medical condition selected from the group consisting of: Type 2 diabetes; Type 1 diabetes; insulin resistance; obesity; NAFLD; NASH; PCOS; a binge eating disorder; a hyperphagic state; hyperphagia; Prader Willi syndrome; an obesity-related genetic disorder; post-bariatric hypoglycemia; iron overload; hereditary hemochromatosis; and combinations of one, two, or more of these.

In some embodiments, the harvest site comprises an anatomical location selected from the group consisting of: the gastro intestinal tract; the mouth; the esophagus; the stomach; the duodenum; the jejunum; the ileum; the colon; an organ; the brain; the lungs; the liver; the bladder; the kidneys; the heart; the intestines; the skin; the peritoneal cavity; and combinations of one, two, or more of these.

In some embodiments, the harvest site comprises an anatomical location selected from the group consisting of: mucosal tissue; submucosal tissue; tissue comprising at least one stem cell; tissue comprising at least one stem cell of the mucosa; tissue comprising at least one mucosal crypt containing a stem cell; mucosal tissue comprising at least one stem-cell containing crypt; organoids; epithelial layer tissue; uroepithelial layer tissue; intestinal epithelial layer tissue; lung epithelial layer tissue; and combinations of one, two, or more of these.

In some embodiments, the deposit site comprises one or more locations in the duodenum and/or proximal jejunum. The system can be configured to treat a medical condition selected from the group consisting of: Type 2 diabetes; Type 1 diabetes; insulin resistance; obesity; NAFLD; NASH; PCOS; a binge eating disorder; a hyperphagic state; hyperphagia; Prader Willi syndrome; an obesity-related genetic disorder; post-bariatric hypoglycemia; recurrent obesity post-bariatric surgery; recurrent metabolic disease post-bariatric surgery; iron overload conditions such as hemochromatosis types 1-4 or bantu siderosis; hereditary hemochromatosis; and combinations of one, two, or more of these.

In some embodiments, the deposit site comprises an anatomical location selected from the group consisting of: the gastro intestinal tract; the mouth; the esophagus; the stomach; the duodenum; the jejunum; the ileum; the colon; an organ; the brain; the lungs; the liver; the bladder; the kidneys; the heart; the intestines; the skin; the peritoneal cavity; and combinations of one, two or more of these.

In some embodiments, the deposit site comprises an anatomical location selected from the group consisting of: luminal wall tissue of GI tract; mucosal tissue of GI tract; submucosal tissue of GI tract; the peritoneal cavity; and combinations of one, two, or more of these.

In some embodiments, the harvesting device comprises a device with multiple needles and/or multiple fluid jets configured to deliver material. The harvesting device can comprise an expandable element, and the multiple needles and/or the multiple fluid jets can be positioned on the expandable element.

In some embodiments, the harvesting device is configured to harvest a tissue sample with a dimension selected from the group consisting of: width of less than 3 mm; a thickness of at least 500 microns and/or at least 600 microns; a thickness of at least 1 mm; a thickness of at least 1.5 mm; and combinations thereof.

In some embodiments, the harvesting device comprises at least one harvesting element. The at least one harvesting element can comprise multiple harvesting elements comprising at least two and/or at least three harvesting elements. The multiple harvesting elements can be configured to harvest tissue simultaneously. The at least one harvesting element can comprise an element selected from the group consisting of: one or more needles; one or more needles to which a vacuum can be applied; one or more biopsy elements; one or more tissue grasping elements; one or more vacuum elements; one or more cutting elements; one or more mucosal lifting elements; one or more dissecting elements; and combinations thereof.

In some embodiments, the harvesting device is configured to obtain tissue samples that preferentially contain pluripotent stem cells. The harvesting device can be further configured to obtain tissue samples that preferentially do not contain terminally differentiated cells of the intestinal mucosa.

In some embodiments, the harvesting device is configured to obtain tissue samples that preferentially do not contain terminally differentiated cells of the intestinal mucosa.

In some embodiments, the harvesting device is configured to obtain tissue samples that contain elements of the local biome of the harvest site.

In some embodiments, the harvesting device is configured to avoid obtaining tissue samples that contain elements of the local biome of the harvest site. The harvesting device can comprise a component configured to reduce resident microbiome population prior to the harvesting of the tissue by the harvesting device.

In some embodiments, the depositing of the material is configured, at locations at and/or proximate the deposit site, to start and/or increase secretion of a material selected from the group consisting of: GLP-1; PYY; GIP; CCK; glicentin; oxyntomodulin; exenatide; exendin 9-39; ghrelin; CCK antagonists; FGF1; FGF19; FGF21; amylin; insulin; leptin; adiponectin; GLP-2; a peptide that engenders insulin sensitization, insulin resistance, and/or satiety; and combinations of one, two, or more of these. The secretions can be started and/or increased relative to a fasting and/or fed state of the patient.

In some embodiments, the depositing of the material is configured, at locations at and/or proximate the deposit site, to stop and/or decrease secretion of a material selected from the group consisting of: GLP-1; PYY; GIP; CCK; glicentin; oxyntomodulin; exenatide; exendin 9-39; ghrelin; CCK antagonists; FGF1; FGF19; FGF21; amylin; insulin; leptin; adiponectin; GLP-2; a peptide that engenders insulin sensitization, insulin resistance, and/or satiety; and combinations of one, two, or more of these. The secretions can be stopped and/or decreased relative to a fasting and/or fed state of the patient.

In some embodiments, the depositing of the material is configured to modify neuronal signaling at locations at and/or proximate the deposit site. The neuronal signaling modification can result in an increase in exenatide and/or GLP-1 secretion. The increased secretions can cause an effect on the patient selected from the group consisting of: an inducement in satiety; a delay in gastric emptying; an increase in pancreatic beta cell mass; and combinations thereof. The neuronal signaling modification can result in an alteration in signaling to the endocrine pancreas. The alteration in signaling to the endocrine pancreas can be configured to perform a function selected from the group consisting of: treat diabetes; improve pancreatic beta cell function; and combinations thereof. The neuronal signaling modification can result in an alteration of autonomic signaling to the liver and/or adipocyte cells. The alteration of autonomic signaling to the liver and/or adipocyte cells can be configured to perform a function selected from the group consisting of: treat fatty liver disease; treat obesity; treat lipodystrophy; and combinations thereof. The neuronal signaling modification can result in an alteration of autonomic signaling to the vasculature. The alteration of autonomic signaling to the vasculature can be configured to perform a function selected from the group consisting of: treat hypertension; treat heart failure; treat diastolic dysfunction; and combinations thereof.

In some embodiments, the depositing of the material is configured to modify neurohormonal signaling in the gastrointestinal tract at locations remote from the deposit site. The material can comprise intestinal stem cells, and the deposit site can comprise one or more locations in the proximal small intestine, and the neurohormonal signaling can result in inducing GLP-1 hormone production in the large intestine and/or in the distal small intestine.

In some embodiments, the material comprises harvested tissue that has been processed. The processing can include amplification of the harvested tissue. The processing can include transgenically modifying the harvested tissue.

In some embodiments, the material further comprises at least one agent. The at least one agent can comprise an agent selected from the group consisting of: a pharmaceutical drug; a nutrient; a hexose; a lipid; an amino acid; a vitamin; a water soluble vitamin; ascorbic acid; a buffering agent; a chemical; a filler; a bio-adhesive agent; a trophic agent; a growth factor and/or other factor used to promote wound healing; a shielding agent; an agent configured to protect one or more components of the deposited material after depositing; and combinations of one, two, or more of these.

In some embodiments, the material further comprises a scaffold and/or other carrier.

In some embodiments, the material further comprises a protecting element configured to protect the material after depositing of the material at the deposit site.

In some embodiments, the material further comprises an adhesive.

In some embodiments, the depositing device comprises multiple needles and/or fluid jets configured to deliver the material. The depositing device can comprise an expandable element, and the multiple needles and/or fluid jets can be positioned on the expandable element.

In some embodiments, the depositing device comprises at least two depositing elements configured to deposit the material at the deposit site. The at least two depositing elements can comprise at least one needle and/or at least one fluid jet. The at least two depositing elements can be configured to deposit the material at the deposit site simultaneously.

In some embodiments, the depositing device comprises at least three depositing elements configured to deposit the material at the deposit site. The at least three depositing elements can comprise at least one needle and/or at least one fluid jet. The at least three depositing elements can be configured to deposit the material at the deposit site simultaneously.

In some embodiments, the system further comprises a processing device configured to process the harvested tissue. The processing device can be configured to amplify cells of the harvested tissue. The amplification can comprise an amplification of cells that can be at least 10 fold, at least 100 fold, and/or at least 1,000,000 fold. The amplification can be performed over a duration of at least 1 day, at least 3 days, at least 15 days, and/or at least 30 days. The material can be deposited at the deposit site within 1 day, within 3 days, within 15 days, within 30 days, and/or within 90 days of the amplification process. The amplified cells can be frozen and subsequently stored. The amplified cells can be stored at a temperature of no more than −80° C., no more than −20° C., and/or no more than 0° C. The freezing of the amplified cells can comprise a rapid freezing process. The processing device can be configured to perform an ex-vivo enriched cell culturing process. The processing device can comprise a medium including trophic factors. The processing device can be configured to perform an encapsulation. The processing device can include a scaffold and/or other cell-carrying component. The processing device can be configured to perform cell sorting. The processing device can be configured to remove collagen from tissue. The processing device can be configured to cause cells to grow into one or more organoids. The processing device can be configured to cause cells to grow into a monolayer sheet of cells. The processing device can be configured to combine the harvested tissue with an agent. The agent can comprise an agent selected from the group consisting of: trophic factor; antioxidant; salicylate; a nonsteroidal anti-inflammatory drug; and combinations thereof. The processing device can be configured to arrange cells in a hydrogel matrix. The hydrogel matrix can be configured to degrade over time after depositing in the patient. The processing device can be configured to genetically, chemically, and/or epigenetically modify the harvested tissue. The processing device can be configured to perform a transgenic modification of the harvested tissue. The transgenic modification can be configured to cause resultant tissue generated by depositing the material to express a material selected from the group consisting of: GLP-1; PYY; GIP; CCK; glicentin; oxyntomodulin; exenatide; exendin 9-39; ghrelin; CCK antagonists; FGF1; FGF19; FGF21; amylin; insulin; leptin; adiponectin; GLP-2; a peptide that engenders insulin sensitization, insulin resistance, and/or satiety; and a combination of one, two, or more of these. The transgenic modification can include Crispr/Cas9 gene editing of the somatic DNA. The transgenic modification can include use of a PiggyBac transposon, lentiviral vector, and/or AAV vector. The processing device can be configured to decontaminate the harvested tissue. The processing device can be configured to alter the microbial content of the harvested tissue. The processing device can comprise antibiotics configured to decontaminate the harvested tissue. The processing device can be configured to confirm that the material does not include infective material. The processing device can be configured to perform a test for endotoxins. The processing device can be configured to reduce microbes. The processing device can be configured to tag tissue. The processing device can comprise a marker such as a fluorescent marker configured to tag tissue. The processing device can comprise an agent that can be added to the harvested tissue. The agent can comprise a bioadhesive agent. The processing device can be configured to treat in-situ tissue of a mammalian subject prior to the harvesting of the tissue by the harvesting device, and the harvested tissue can comprise at least a portion of the treated in-situ tissue. The processing device can comprise one or more scaffolds. The one or more scaffolds can comprise one or more cellular scaffolds. The one or more scaffolds can comprise an acellular scaffold. The one or more scaffolds can comprise tissue of the small intestine of a mammal. The one or more scaffolds can comprise porcine small intestinal submucosa and/or a gelatinous protein mixture.

In some embodiments, the system further comprises a treatment device. The treatment device can comprise at least a tissue treatment device. The tissue treatment device can comprise a tissue ablation device. The treatment device can further comprise a tissue expansion device. The treatment device can comprise an agent delivery device. The treatment device can be configured to treat tissue at an anatomical location selected from the group consisting of: the gastro intestinal tract; the mouth; the esophagus; the stomach; the duodenum; the jejunum; the ileum; the colon; an organ; the brain; the lungs; the liver; the bladder; the kidneys; the heart; the intestines; the skin; the peritoneal cavity; and combinations of one, two, or more of these.

In some embodiments, the system further comprises an agent configured to be delivered to the patient and/or the mammalian subject. The agent can be configured to be delivered to the mammalian subject. The agent can be configured to be delivered to the patient. The agent can comprise an agent selected from the group consisting of: pharmaceutical drug; antibiotic; probiotic; prebiotic; iron; anti-inflammatory agent; NSAID; immunosuppressant; and combinations of one, two, or more of these.

In some embodiments, the system further comprises an access device for introducing at least the harvesting device into the mammalian subject. The access device can comprise a device selected from the group consisting of: endoscope; an endoscope-attached sheath; a laparoscopic port; a vascular introducer; and combinations of one, two, or more of these.

In some embodiments, the system further comprises at least one functional element. The at least one functional element can be positioned in the harvesting device and/or the depositing device. The at least one functional element can comprise a sensor. The at least one functional element can comprise a transducer.

According to another aspect of the present inventive concepts, a method of treating a medical condition of a patient; selecting a patient; harvesting tissue from a harvest site of a mammalian subject; and depositing material at a deposit site of a patient. The deposited material is based on the harvested tissue, and the deposited material is configured to generate resultant tissue configured to treat the medical condition of the patient.

In some embodiments, the patient comprises the mammalian subject.

In some embodiments, the patient comprises a different mammal than the mammalian subject.

In some embodiments, the method further comprises performing a procedure prior to harvesting the tissue. The procedure can be performed on the patient and/or the mammalian subject. The procedure can comprise a procedure selected from the group consisting of: diet; delivery of an agent; tissue treatment procedure; and combinations of one, two, or more of these.

In some embodiments, the method further comprises processing the harvested tissue.

In some embodiments, the method further comprises performing a procedure on the patient prior to depositing the material. The procedure can be performed after the harvesting of tissue from the mammalian subject.

In some embodiments, the method further comprises performing a procedure on the patient after depositing the material in the patient.

According to another aspect of the present inventive concepts, a method of treating a medical condition of a patient; selecting a patient; and depositing a generated material at a deposit site of a patient. The deposited material is configured to generate resultant tissue configured to treat the medical condition of the patient.

In some embodiments, the method further comprises performing a transgenic modification of a starting material to create the generated material.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a system for depositing material at a deposit site of a patient, consistent with the present inventive concepts.

FIG. 2 is a flow chart of a method for depositing material at a deposit site of a patient, consistent with the present inventive concepts.

FIG. 3 is a flow chart of another method for depositing material at a deposit site of a patient, consistent with the present inventive concepts.

FIG. 4 is a flow chart of another method for depositing material at a deposit site of a patient, consistent with the present inventive concepts.

### DETAILED DESCRIPTION OF THE INVENTION

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one, two, or more of these.

As used herein, the term “proximate” shall include locations relatively close to, on, in and/or within a referenced component, anatomical location, or other location. As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

In this specification, unless explicitly stated otherwise, “and” can mean “or,” and “or” can mean “and.” For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold. within a threshold range of values and/or outside a threshold range of values, to cause a desired effect (e.g. efficacious therapy) and/or to prevent or at least reduce the effects of (hereinafter “prevent” or “reduce”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. “Positive pressure” includes pressure above room pressure or simply a pressure that is greater than another pressure, such as a positive differential pressure across a fluid pathway component such as a valve. “Negative pressure” includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function. In some embodiments, a functional element comprises one or more elements constructed and arranged to perform a function selected from the group consisting of: deliver energy; extract energy (e.g. to cool a component); deliver a pharmaceutical drug or other agent; manipulate a system component or patient tissue; record or otherwise sense a parameter such as a patient physiologic parameter or a patient anatomical parameter; and combinations of one or more of these. A functional element can comprise a fluid, such as an ablative fluid (as described hereabove) comprising a liquid or gas configured to ablate or otherwise treat tissue. A functional element can comprise a reservoir, such as an expandable balloon configured to receive an ablative fluid. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, thermal energy such as heat energy and/or cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one, two, or more of these.

As used herein, the term “fluid” can refer to a liquid, gas, gel, and/or any flowable material, such as a material which can be propelled through a lumen and/or opening.

As used herein, the term “tissue modification procedure” refers to a procedure performed on tissue to modify a property of the tissue treated and/or tissue proximate the tissue treated (“treated tissue” herein). A tissue modification procedure can result in necrosis and/or removal of tissue, after which “replacement tissue” develops in the place of the removed tissue, the replacement tissue having different properties than the tissue that was removed. A tissue modification procedure can result in a reduction in surface area of the treated tissue (e.g. a reduction in the luminal surface area of an inner wall of tubular tissue), such as to modify secretions and/or absorptions of the tissue. A tissue modification procedure can include: delivery of energy to tissue (e.g. delivery of ablative heat, ablative cold, and/or ablative electromagnetic energy); mechanical removal and/or disruption of tissue; chemical ablation of tissue; and combinations of one, two, or more of these. Treated tissue or replacement tissue (“treated tissue” herein) can have modified properties including but not limited to: modification of one or more absorptive properties of the tissue; modification of one or more secretive properties of the tissue; modification of neuronal signaling of the tissue; and combinations of one, two or more of these. Effects of the tissue modification procedure can occur acutely and/or it can take place over time, such as days, weeks or months. In some embodiments, a tissue modification procedure includes injecting one or more materials into the submucosal tissue of a GI lumen, such as to expand a submucosal tissue layer, for example a full (360°) or partial circumferential expansion of an axial segment of the GI tract. These tissue expansion procedures can cause a relatively acute result (e.g. less than 30 minutes), such as to perform a subsequent mucosal ablation procedure wherein the expanded tissue acts as a safety margin of tissue during the ablation, protecting the underlying layers from damage. In some embodiments, the full or partial circumferential submucosal tissue expansion is performed to cause luminal narrowing at the axial segment, such as is described in applicant's co-pending application U.S. patent application Ser. No. 15/156,585 (Attorney Docket No. 41714-711.301, Client Docket No. MCT-024-US), entitled “Systems, Devices and Methods for the Creation of a Therapeutic Restriction in the Gastrointestinal Tract”, filed May 17, 2016, the contents of which are incorporated herein by reference in its entirety. This luminal narrowing can be configured to last for a prolonged period of time, such as at least 1 day, at least 1 week, and/or at least one month. This luminal narrowing can be performed to restrict food intake of the patient, and/or for other purposes.

As used herein, the term “ablative temperature” refers to a temperature at which tissue necrosis or other desired tissue treatment occurs (e.g. a temperature sufficiently hot or sufficiently cold to cause tissue necrosis or any desired effect). As used herein, the term “ablative fluid” refers to one or more liquids, gases, gels or other fluids whose thermal properties cause tissue necrosis and/or another desired tissue treatment (e.g. one or more fluids at an ablative temperature). Alternatively or additionally, “ablative fluid” refers to one or more fluids whose chemical properties (at room temperature, body temperature or otherwise) cause tissue necrosis or another desired tissue treatment. A treatment element (e.g. a functional element) of the present inventive concepts can comprise one or more ablative fluids and/or comprise one or more elements that deliver one or more ablative fluids (e.g. deliver the fluids onto a tissue surface and/or into a volume of tissue).

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems and methods for implanting or otherwise depositing material at a deposit site of a patient (e.g. a mammalian patient), such as to provide a therapeutic benefit to the patient. The systems and methods can be configured to treat a medical condition of the patient, such as one, two, three or more diseases, disorders, and/or other medical conditions of a patient. The system includes a depositing device for the depositing material at a deposit site, such as material that is based on tissue harvested at a harvest site with a harvesting device of the system. The deposited material generates tissue that is configured to treat the medical condition of the patient.

Referring now to FIG. 1, a schematic view of a system for depositing a material at a deposit site of a patient is illustrated, consistent with the present inventive concepts. System 10 includes depositing device 600. Depositing device 600 comprises a device for implanting, placing, seeding, inserting, spraying, topically applying, and/or otherwise depositing (“depositing” herein) material, such as material 60 described herebelow, at a “deposit site” of a patient. The deposit site can comprise one, two, or more sites on and/or within a patient (e.g. on the patient's skin and/or within the body of the patient, respectively). After the depositing of material 60, new tissue is generated at the deposit site and locations proximate the deposit site. The new tissue (including material 60 and/or void of material 60) comprises “resultant tissue” herein. Properties of the resultant tissue can be driven by or otherwise based on material 60 (e.g. properties including one or more proteins that are expressed by the resultant material). Generation of the resultant tissue by the systems and methods of the present inventive concepts can provide a therapeutic benefit used to treat one or more medical conditions of the patient.

In some embodiments, system 10 includes a device for harvesting tissue of the patient, harvesting device 400, which harvests tissue, tissue 61 described herebelow, at a “harvest site” of the patient. The harvest site can include one, two, or more patient tissue locations such as one or more locations on the skin of the patient, and/or one or more locations within the patient's body. In some embodiments, system 10 includes a device, processing device 500, that is configured to process tissue 61. Processing device 500 comprises one or more elements for processing tissue 61, processing element 550 shown. Processing device 500 can be used to process tissue 61 after its harvest by harvesting device 400. In some embodiments, processing device 500 is configured to treat in-situ tissue prior to its harvest. In some embodiments, system 10 includes treatment device 700. Treatment device 700 is configured to treat tissue at a “treatment site” of the patient. The treatment site can include one or more locations on the skin of the patient, and/or one or more locations within the patient's body. In some embodiments, treatment device 700 treats tissue of a deposit site and/or tissue proximate a deposit site, such as when performing a tissue treatment procedure prior to the depositing of material 60 at a deposit site.

In some embodiments, system 10 is configured to provide a treatment as described herebelow in reference to FIGS. 2, 3, and/or 4. System 10 can be configured to treat one, two, three, or more medical conditions selected from the group consisting of: Type 2 diabetes; Type 1 diabetes; Double diabetes; gestational diabetes; hyperglycemia; pre-diabetes; monogenic diabetes; maturity onset diabetes of the young; impaired glucose tolerance; insulin resistance; hyperinsulinemia; hypoinsulinemia; non-diabetic hypoglycemia; elevated albuminuria; non-alcoholic fatty liver disease; non-alcoholic steatohepatitis; obesity; obesity-related disorder; polycystic ovarian syndrome; hypertriglyceridemia; hypercholesterolemia; psoriasis; gastroesophageal reflux disease; coronary artery disease; stroke; transient ischemic attack; cognitive decline; dementia; Alzheimer's Disease; neuropathy; diabetic nephropathy; retinopathy; heart disease; diabetic heart disease; heart failure; diabetic heart failure; hirsutism; hyperandrogenism; fertility issues; menstrual dysfunction; cancer; liver cancer; ovarian cancer; breast cancer; endometrial cancer; cholangiocarcinoma; adenocarcinoma; glandular tissue tumor; stomach cancer; colorectal cancer; prostate cancer; diastolic dysfunction; hypertension; myocardial infarction; microvascular disease related to diabetes; anorexia nervosa; anorexia; a binge eating disorder; a hyperphagic state; hyperphagia; polyphagia; Prader Willi syndrome; an obesity-related genetic disorder; hypoglycemia; hypoglycemia that presents after a bariatric procedure (referred to as “post-bariatric hypoglycemia”); recurrent obesity post-bariatric surgery; recurrent metabolic disease post-bariatric surgery; iron overload conditions such as hemochromatosis types 1-4 and/or bantu siderosis; hypercholesterolemia; pancreatic cancer; short bowel syndrome; sleep apnea; arthritis; rheumatoid arthritis; and combinations of these.

Disorders of the intestine, such as celiac disease and inflammatory bowel disease, have significant morbidity and mortality for patients around the world. Intestinal procedures in these patients are currently limited to diagnostic procedures, such as mucosal biopsy, or palliative interventional procedures. System 10 can be configured to treat various disorders of the intestine, such as celiac disease and/or inflammatory bowel disease. However, findings by applicant into the role of the mucosa in these diseases, coupled with processes for gene therapy and cell therapy, enable system 10 to provide novel therapies for patients with these diseases.

In some embodiments, material 60 includes cellular material that can engraft to the deposit site and generate resultant tissue, such as when the deposited material 60 divides, such as through symmetric and/or asymmetric cell division. Progenitor cells in material 60 (e.g. stem cells that can be selected through an enrichment process, such as an enrichment process performed on previously harvested tissue 61) can be used to repopulate and/or reconstitute a neo-mucosa, such as a neomucosa with differentiating cells that form a mucosal epithelium at the deposit site and proximate locations (“deposit site” herein). Symmetric division of progenitor cells expand laterally along the surface of the deposit site to form a contiguous epithelial layer. In these embodiments, the resultant tissue (e.g. neo-mucosa) can provide one or more barrier functions, absorptive functions, and/or secretory functions, such as to provide an endocrine function and/or a neuro-endocrine function. These one or more functions can be different than those of the tissue previously present at and/or proximate the deposit site. The absorptive cells in the resultant tissue can enable the absorption of nutrients into the body, such as glucose, amino acids, cholesterol, and the like, for example absorption of a new nutrient and/or a modified (increased or decreased) absorption of a nutrient (such as iron). The secretory function can enable hormonal signaling from this portion of the patient's anatomy (e.g. the patient's intestine) to other body locations, for example a new hormonal signaling and/or modified (increased or decreased) hormonal signaling. For example, the hormonal signaling can modify and/or modulate pancreatic endocrine function (such as the production of insulin and glucagon), pancreatic exocrine function (such as the production of pancreatic digestive enzymes), and the body's insulin resistance, particularly liver insulin resistance. The barrier function provided by the resultant tissue can comprise a barrier function that is different than the barrier function performed by the tissue (e.g. mucosal tissue) of the deposit site prior to the procedure, such as a barrier function that provides different passage of nutrients and/or gut microbiota.

The intestinal lining serves as a barrier function to prevent infectious agents from being transported from the lumen of the gastrointestinal tract into the body. It also serves as an absorptive layer to permit the passage of nutrients, minerals, peptides, fuel (sugars, protein, fat), and bile acids (e.g. in particular portions of the GI tract). The intestinal lining also serves as a signaling organ that communicates signals from the gastrointestinal surface to the rest of the body through neuronal and/or hormonal signals. Different segments of the intestinal lining exhibit different properties in their capacity to absorb different materials as well as in their signaling properties in the fasting and fed states. The harvest site and the deposit site can have similar function in their capacity to serve as a barrier to infection, but may or may not exhibit differences in their absorptive or signaling properties between them.

System 10 can be used to cause resultant tissue (proximate the deposit site) to exhibit one or more properties of the tissue of the harvest site, as described immediately hereabove. The resultant tissue properties can provide a therapeutic benefit to the patient, as described herein. Alternatively or additionally, tissue can be harvested (from any location including the deposit site location), modified ex-vivo (e.g. using processing device 500), such that the resultant tissue proximate the deposit site exhibits one or more properties based on the harvest site and/or the ex-vivo modification. Similarly, this resultant tissue can have properties that provide a therapeutic benefit to the patient.

Material 60 can be deposited at one or more deposit sites to modify the secretions delivered at and/or proximate that deposit site. For example, material 60 can include tissue 61 that is harvested from the terminal ileum, colon, and/or other location, that results, after deposit at a deposit site such as the duodenum and/or proximal jejunum, in increased secretions of peptides that engender insulin sensitization, insulin resistance, and/or satiety (where increased secretions shall include an increase from no secretion of that peptide prior to depositing of material 60). In some embodiments, material 60 be configured to start and/or increase the secretion of (herein “increase the secretion of”) one or more of: GLP-1; PYY; GIP; CCK; glicentin; oxyntomodulin; exenatide; exendin 9-39; ghrelin; CCK antagonists; FGF1; FGF19; FGF21; amylin; insulin; leptin; adiponectin; GLP-2; and/or a peptide that engenders insulin sensitization, insulin resistance, and/or satiety. The secretion of these hormones and/or peptides from the resultant tissue to the patient's body can be timed relative to fasting and/or fed states of the patient, such as to improve treatment of the one or more medical conditions of the patient. In this way, the resulting hormonal changes in the patient's body are different than before material 60 was deposited (e.g. differences representing the quantity and/or the timing of the secretions).

In some embodiments, depositing of material 60 is configured to stop or at least reduce (herein “reduce”) the secretion of one or more of: GLP-1; PYY; GIP; CCK; glicentin; oxyntomodulin; exenatide; exendin 9-39; ghrelin; CCK antagonists; FGF1; FGF19; FGF21; amylin; insulin; leptin; adiponectin; GLP-2; a peptide that engenders insulin sensitization, insulin resistance, and/or satiety; and combinations of one, two, or more of these.

Material 60 can be deposited at one or more deposit sites to modify the absorptions that occur at and/or proximate the deposit site, such as to modify the absorption of one or more of: nutrients; fat; protein; glucose; fructose; iron; cholesterol; minerals; peptides; bacteria; virus; fungus; bile salts; bile acids; enzymes (e.g. digestive enzymes produced by the pancreas); micronutrients; macronutrients; pharmacologic agents; alcohol; water; fluids; salt; and/or electrolyte solutions. Treatment could modify absorption for treated area as well as distal locations in intestine (e.g. modify jejunal and/or ileal absorption by modifying absorption in duodenum).

For example, diarrhea can be treated, such as by increasing absorption of water, electrolyte solutions, and/or other fluids in the small intestine and/or large intestine.

For example, iron absorption could be modified (e.g. reduced), such as to treat an iron overload disorder. In some embodiments, depositing of material 60 is configured to reduce the absorption of one or more nutrients, such as iron.

For example, fat and/or cholesterol absorption could be modified (e.g. reduced), such as a modification in the absorptions that subsequently occur in the distal small intestine, such as to treat a lipid disorder (e.g. hypercholesterolemia).

For example, vitamin B12 absorption could be modified (e.g. increased), such as a modification in the B12 absorptions that occur in patients with pernicious anemia.

For example, chronic gastrointestinal wounds could be treated (e.g. healed), such as by introducing a new population of stem cells to populate the wounded area.

Material 60 can be deposited at one or more deposit sites to modify the neuronal signaling that occurs at and/or proximate the deposit site. For example, an exenatide and/or GLP-1 secretion increase can be generated to induce satiety, delay gastric emptying, increase pancreatic beta cell mass, and/or cause other physiological changes (e.g. at least partially through neuronal mechanisms). For example, an alteration of signaling to the endocrine pancreas can be generated, such as to treat diabetes and/or improve pancreatic beta cell function in patients with impaired beta cell response to glucose. For example, an alteration of autonomic signaling to the liver and/or adipocyte cells can be generated, such as to treat fatty liver disease and/or obesity or lipodystrophy, respectively. For example, an alteration of autonomic signaling to the vasculature can be generated, such as to treat hypertension, heart failure, and/or diastolic dysfunction. Hormonal signaling may also be altered elsewhere in the gastrointestinal tract, at locations remote from the deposit site, through neurohormonal signaling, such as by inducing GLP-1 hormone production in the large intestine and/or the distal small intestine, after depositing material 60 comprising intestinal stem cells at one or more deposit sites in the proximal small intestine.

In some embodiments, depositing of material 60 is configured to modify immune reaction at locations proximate and/or remote from the deposit site. For example, depositing of material 60 can be configured to produce and/or secrete a substance (e.g. an antibody, RNA aptamer, and/or an enzyme) that binds to an antigen known to trigger an autoimmune response in the patient, (e.g. such as an antigen that triggers gluten sensitivity and/or a gluten allergy) such that the depositing of material 60 serves to prevent the patient from experiencing an immune reaction, such as to prevent the patient experiencing a gluten allergy or sensitivity (e.g. for a patient with Celiac disease).

Depositing device 600, harvesting device 400, and/or treatment device 700 (singly or multiply, “devices 600, 400, 700”) can each be configured to be introduced into the body via a natural orifice (e.g. via the mouth or rectum), or via a skin incision (e.g. in an open surgical procedure or a minimally invasive surgical procedure).

Devices 600, 400, 700, can comprise a catheter configuration, such as a catheter that includes an elongate flexible shaft. The shaft can comprise an insertable length configured to access the duodenum, the jejunum, and/or the ileum, such as when placed via the patient's mouth, such as a length of at least 100 cm, 130 cm, 150 cm, respectively. In some embodiments, harvesting device 400 comprises a longer length than depositing device 600, such as when harvesting device 400 accesses the ileum (e.g. via the mouth) and depositing device 600 accesses the duodenum and/or jejunum (e.g. via the mouth). In some embodiments, harvesting device 400 comprises a shorter length than depositing device 600, such as when harvesting device 400 accesses the colon (e.g. via the rectum) and depositing device 600 accesses the duodenum and/or jejunum (e.g. via the mouth). In some embodiments, harvesting device 400 comprises a shorter length than depositing device 600, such as when harvesting device 400 accesses the duodenum (e.g. via the mouth) and depositing device 600 accesses the jejunum (e.g. via the mouth).

Devices 600, 400, 700 can be configured to be introduced into the patient: through an endoscope (e.g. through a working channel of an endoscope); alongside an endoscope (e.g. through a scope-attached sheath and/or over a guidewire); through a laparoscopic port; and/or via another body access device, such as access device 50 described herebelow.

Depositing device 600 can comprise one, two, or more devices configured to deposit material 60 at a deposit site of a patient. Depositing device 600 comprises one or more elements for depositing material 60, depositing element 650 shown. Depositing device 600 can include two or more devices that are similar and/or dissimilar (e.g. when a first depositing device 600 and a second depositing device 600 comprise dissimilar lengths, and/or dissimilar depositing elements 650). The material deposited by depositing device 600, material 60 shown in FIG. 1 and described herebelow, can include tissue 61, processed tissue (e.g. tissue 61 processed as described herein), an agent (e.g. a pharmaceutical agent or other agent 62 as described herein), and/or other material (e.g. a material configured to provide and/or support a diagnostic or therapeutic benefit).

Material 60 can be deposited at one, two, or more deposit sites of a patient. Deposit sites can include, but are not limited to: luminal wall tissue of gastrointestinal (GI) tract; mucosal tissue of GI tract; and/or submucosal tissue of GI tract; and/or the peritoneal cavity. Deposit sites can include but are not limited to: the gastro intestinal tract; the mouth; the esophagus; the stomach; the duodenum; the jejunum; the ileum; the colon; an organ; the brain; the lungs; the liver; the bladder; the kidneys; the heart; the intestines; the skin; and/or the peritoneal cavity.

In some embodiments, depositing element 650 comprises at least two depositing elements 650a,b, and/or at least three depositing elements 650 a,b,c. The multiple depositing elements 650 can be configured to deposit material 60 simultaneously and/or sequentially. Depositing element 650 can comprise one, two, three, or more needles through which material 60 can be deposited at one or more deposit sites. Alternatively or additionally, depositing element 650 can comprise one, two, three, or more fluid jets through which material 60 is deposited at one or more deposit sties. In some embodiments, depositing element 650 comprises one or more material 60 depositing elements positioned on an expandable element, such as an inflatable balloon, a flexible basket or cage, a series of radially deployable arms, and/or an unfurlable sheet. In some embodiments, depositing device 600 is configured to lift tissue (e.g. expansion of submucosal tissue via injection into the submucosa of a balanced salt solution such as normal saline), prior to the depositing of material 60.

In some embodiments, depositing device 600 is configured to maintain (e.g. to protect) material 60 at the deposit location (e.g. prevent the migration of material 60 from the deposit site). For example, depositing device 600 can include a gel configured to be applied on top of the delivered material 60 and/or a sleeve configured to be placed over the material 60 (e.g. a sleeve placed in a lumen whose wall has received material 60).

In some embodiments, material 60 includes a carrier element, carrier 63 described herein, such as an adhesive, clip, stent, tubular structure, and/or other carrier element, and depositing device 600 deploys carrier 63 to deposit material 60.

In some embodiments, depositing device 600 is configured to deposit material 60 along one or more deposit sites with a cumulative length of at least 25 mm. In some embodiments, material 60 is deposited within a cumulative surface area (e.g. surface area of the inner wall of one or more segments of the GI tract) of at least 50 cm², at least 100 cm², or at least 250 cm² (e.g. material is deposited into one or more “patches” that cover 1% to 100% of that surface area).

In some embodiments, depositing device 600 is of similar construction arrangement to a device described in applicant's co-pending application U.S. Provisional Patent Application Ser. No. 62/533,569 (Attorney Docket No. 41714-715.101, Client Docket No. MCT-025-PR1), entitled “Intestinal Catheter Device and System”, filed Jul. 17, 2017, the contents of which is incorporated herein by reference in its entirety for all purposes.

Harvesting device 400 can comprise one, two, or more devices configured to harvest tissue 61 at a harvest site of a patient. Harvesting device 400 comprises one or more elements for harvesting tissue 61, harvesting element 450 shown. Harvesting device 400 can include two or more devices that are similar and/or dissimilar (e.g. when a first harvesting device 400 and a second harvesting device 400 comprise dissimilar lengths and/or dissimilar harvesting elements 450). Harvesting device 400 can comprise a device similar to a device used to perform endoscopic mucosal resection (EMR) procedures and/or endoscopic submucosal dissection (ESD) procedures. Harvesting device 400 can be configured to obtain one, two, or more tissue 61 samples. Harvesting device 400 can be configured to perform a core or punch biopsy of tissue (e.g. mucosal tissue). In some embodiments, harvesting device 400 is configured to simultaneously obtain multiple samples of tissue 61 (e.g. multiple simultaneous core and/or punch biopsies). In some embodiments, harvesting device 400 comprises a suction and/or guillotine biopsy device. In some embodiments, harvesting device 400 comprises a device configured to scrape a tissue surface, such as to harvest tissue of the mouth or other body location.

Harvesting device 400 can be configured to obtain individual tissue samples with one or more dimensions selected from the group consisting of: a width of less than 3 mm; a thickness (e.g. tissue depth) of at least 500 microns or at least 600 microns; a thickness of at least 1 mm; a thickness of at least 1.5 mm; and combinations of one, two, or more of these.

Harvesting element 450 can comprise one, two, or more elements configured to capture tissue, such as one or more: needles (e.g. one or more needle to which a vacuum can be applied); biopsy elements; tissue grasping elements; vacuum elements; cutting elements; mucosal lifting elements, and/or dissecting elements. In some embodiments, harvesting element 450 comprises at least two harvesting elements 450a,b, and/or at least three harvesting elements 450 a,b,c. The multiple harvesting elements 450 can be configured to harvest tissue simultaneously and/or sequentially.

In some embodiments, harvesting device 400 is of similar construction and arrangement to a device described in applicant's co-pending application U.S. Provisional Patent Application Ser. No. 62/533,569 (Attorney Docket No. 41714-715.101, Client Docket No. MCT-025-PR1), entitled “Intestinal Catheter Device and System”, filed Jul. 17, 2017, the contents of which is incorporated herein by reference in its entirety for all purposes.

Tissue 61 can comprise material harvested from one, two, or more anatomical locations of a patient. Anatomical locations for harvest sites include but are not limited to: the gastro intestinal tract; the mouth; the esophagus; the stomach; the duodenum; the jejunum; the ileum; the colon; an organ; the brain; the lungs; the liver; the bladder; the kidneys; the heart; the intestines; the skin; and/or the peritoneal cavity. Tissue 61 can comprise autograft tissue, autogenous tissue, autologous tissue, allograft tissue, and/or xenograft tissue.

Tissue 61 (e.g. tissue 61 captured by harvesting device 400) can comprise: mucosal tissue; submucosal tissue; tissue comprising at least one stem cell; tissue comprising at least one stem cell of the mucosa; tissue comprising at least one mucosal crypt containing a stem cell; mucosal tissue comprising at least one stem-cell containing crypt; organoids; epithelial layer tissue; uroepithelial layer tissue; intestinal epithelial layer tissue; and/or lung epithelial layer tissue.

In some embodiments, harvesting device 400 is configured to harvest tissue 61 (e.g. obtain individual tissue samples) that preferentially contain pluripotent stem cells and/or preferentially do not contain terminally differentiated cells of the intestinal mucosa. In some embodiments, harvesting device 400 is configured to harvest tissue 61 (e.g. obtain individual tissue samples) that also contain elements of the local microbiome of the harvest site. Alternatively, harvesting device 400 can be configured to harvest tissue 61 that preferentially do not contain elements of the local microbiome of the harvesting location, such as by harvesting tissue 61 that has been pre-treated by a component of system 10 (e.g. pre-treated by a component of harvesting device 400) to reduce its resident microbiome population (e.g. a component that disinfects the tissue to be harvested).

Tissue 61 can comprise hormonal activating tissue. Alternatively or additionally, tissue 61 can comprise hormonal deactivating tissue.

In some embodiments, tissue 61 does not include tissue of (e.g. harvesting device 400 avoids harvesting tissue from): the lower esophageal sphincter; the pylorus; the ampulla of Vater; the ileocecal valve; and combinations of one, two, or more of these.

Tissue 61 can comprise captured tissue that has at least 1 cm² of surface area (e.g. at least 1 cm² of mucosal tissue luminal surface area), which can be harvested by harvesting device 400 in a single step or multiple steps. The term “surface area”, when used to describe the surface area of a segment of sampled tissue that has a relatively flat geometry (e.g. a length and width much greater than its thickness), is to be taken as the surface area of one side of the sample. When used to describe the surface area of multiple samples, each with a relatively flat geometry, “surface area” is to be taken as the cumulative surface areas of a single side of each sample.

In some embodiments, tissue 61 comprises a minimum and/or maximum amount of tissue to be harvested, such as an amount selected from the group consisting of: at least one core biopsy; no more than 20 core biopsies; at least 1000 cells; no more than 1 billion cells; and combinations of one, two, or more of these.

Treatment device 700 can comprise one, two, or more devices configured to ablate, remove, modify, expand, and/or otherwise treat tissue at a treatment site of a patient. Treatment device 700 comprises one or more elements for treating tissue of the patient, treatment element 750 shown. Treatment device 700 can include two or more devices that are similar and/or dissimilar (e.g. when a first treatment device 700 and a second treatment device 700 comprise dissimilar lengths and/or dissimilar treatment elements 750). In some embodiments, treatment device 700 comprises a first treatment device 700a that causes tissue to necrose (e.g. via delivery of thermal energy (e.g. heat energy and/or cryogenic energy), electrical energy, and/or a chemical agent to tissue), and a second treatment device 700b that provides an abrasive force to the necrosed tissue. The second treatment device can be used in the same clinical procedure as treatment device 700a is used, or in a subsequent clinical procedure (e.g. a second clinical procedure 1, 2, or more days after the first clinical procedure). In these embodiments, tissue can be removed proximate (e.g. at and/or near) one or more deposit sites at which material 60 is to be deposited (e.g. deposited using depositing device 600).

The tissue treated by treatment device 700 can include mucosal tissue; submucosal tissue; tissue comprising at least one stem cell; tissue comprising at least one stem cell of the mucosa; tissue comprising at least one mucosal crypt containing a stem cell; and/or mucosal tissue comprising at least one stem-cell containing crypt. Tissue treated by treatment device 700 can include tissue of one or more anatomical locations selected from the group consisting of: the gastro intestinal tract; the mouth; the esophagus; the stomach; the duodenum; the jejunum; the ileum; the colon; an organ; the brain; the lungs; the liver; the bladder; the kidneys; the heart; the intestines; the skin; the peritoneal cavity; and combinations of one, two, or more of these. Tissue treated by treatment device 700 can comprise tissue that activates and/or deactivates hormonal signals and/or signaling pathways. In some embodiments, tissue treated by treatment device 700 does not include tissue of (e.g. treatment device 700 avoids adversely effecting tissue from): the lower esophageal sphincter; the pylorus; the ampulla of Vater; the ileocecal valve; and combinations of one, two, or more of these.

Treatment device 700 can be used to perform a tissue modification procedure as defined hereabove, such as a tissue modification procedure performed proximate (e.g. at and/or near) one or more intended deposit sites for material 60. In some embodiments, treatment device 700 is configured to remove tissue from the deposit site (e.g. prior to the depositing of material 60), such that resultant tissue (regrowth of tissue after the treatment performed by treatment device 700), includes tissue properties that are “driven by” the characteristics of material 60. In these embodiments, the removal of tissue can reduce the effects (e.g. competing effects) of the tissue previously present at the deposit site (e.g. tissue removed using treatment device 700).

In some embodiments, a tissue modification procedure is performed using treatment device 700 at a location distal to a deposit site and/or proximal to a deposit site. In these embodiments, a tissue modification procedure may or may not also be performed at the deposit site.

In some embodiments, a tissue modification procedure is performed proximal to the deposit site (e.g. upstream in the GI tract), such as to protect the deposited material (e.g. by reducing the intraluminal contents from overexposing the site, such as for a period of 2 weeks in which food intake is limited). For example, treatment device 700 can be configured to perform a luminal narrowing procedure in which one or more materials are injected into a full or partial circumferential portion of an axial segment of the GI tract, as described herein, such as to restrict the patient's food intake and/or to modify the flow of contents within the lumen of the GI tract. Alternatively or additionally, a tissue modification procedure can be performed distal to the deposit site (e.g. downstream in the GI tract). For example, treatment device 700 can be used to perform a luminal narrowing procedure at an axial segment of the GI tract downstream from the deposit site, such as to cause the deposit site to be washed or bathed by contents passing therethrough (e.g. washing or bathing that results from intraluminal contents remaining at the deposit site for a longer period of time due to the downstream narrowed segment).

Treatment element 750 can comprise one, two, three, or more treatment elements configured to ablate, remove, resurface, denature, and/or otherwise effect tissue, such as mucosal tissue. Treatment element 750 can deliver an ablative fluid to treat the tissue (e.g. an ablative fluid applied directly to the tissue or delivered to a balloon placed in contact with tissue). Treatment element 750 can deliver energy to tissue, such as electrical energy; magnetic energy; chemical energy; sound energy; and/or light energy. In some embodiments, treatment element 750 comprises multiple treatment elements arranged in a circumferential pattern and/or a single element that treats a circumferential segment of tubular tissue (e.g. delivers energy and/or an agent to the full circumferential wall of a segment of intestine). The depositing of material 60 can occur before and/or after the use of treatment element 750, such as by injecting material 60 into the submucosa and subsequently performing a treatment with treatment element 750, and/or by performing a treatment with element 750 and then depositing material 60. Treating and depositing steps can be performed in the same procedure or in different procedures. These steps can be performed within minutes of one another, within 3 days, and/or within 5 days.

In some embodiments, treatment device 700 is of similar construction arrangement to a device described in applicant's co-pending application U.S. Provisional Patent Application Ser. No. 62/533,569 (Attorney Docket No. 41714-715.101, Client Docket No. MCT-025-PR1), entitled “Intestinal Catheter Device and System”, filed Jul. 17, 2017, the contents of which is incorporated herein by reference in its entirety for all purposes.

System 10 can further comprise a device configured to provide access within the patient, access device 50. Access device 50 can comprise an endoscope, an endoscope-attached sheath, a laparoscopic port, a vascular introducer, and/or another patient access device. Access device 50 can further comprise one or more guidewires, e.g. one or more guidewires over which devices 600, 400, and/or 700 are introduced into the patient (e.g. and subsequently withdrawn from the patient), such as by using standard “over the wire” clinical techniques. Access device 50 can comprise a camera, such as camera and a display, such as when access device 50 comprises an endoscope.

In some embodiments, material 60 comprises tissue 61 that is not processed. Alternatively or additionally, material 60 can comprise processed tissue 61, such as when tissue 61 is processed by processing device 500. The term “material 60”, as used herein, shall include a partially processed material 60, such as a material that is to be further processed prior to implantation in the patient at the deposit site. The term “material 60”, as used herein, shall include “resultant material”, as defined herein.

Processing device 500 can be configured to extract, isolate, separate, and/or otherwise collect particular cells from tissue 61, such as stem cells.

Processing device 500 can be configured to generate new cells, such as to amplify the number of cells in a sample (e.g. amplify the number of stem cells). Processing device 500 can be configured to generate an amplified volume of tissue 61, such as an amplification of at least 10 fold, at least 100 fold, and/or at least 1,000,000 fold the number of cells harvested. This amplification process can be performed over a duration of least 1 day, 3 days, 15 days, and/or 30 days. Material 60 generated by the amplification process can be deposited in the patient within 1 day, 3 days, 15 days, 30 days, and/or 90 days of the amplification process. Alternatively, material 60 can be stored indefinitely, and eventually deposited in the patient at any point after the amplification process. Processing device 500 (or another component of system 10) can include a freezing component, wherein material 60 is frozen (e.g. and stored at less than 0° C., such as storage at a temperature less than −20° C., less than −80° C., or at a temperature of approximately −200° C.). In some embodiments, material 60 is rapidly frozen (e.g. flash-frozen in less than 5 seconds, such as freezing using liquid nitrogen) such as to optimize viability of a majority of the tissue.

Processing device 500 can be used to collect stem cells (e.g. intestinal stem cells) from tissue 61 comprising the mucosa (e.g. the deep mucosa), submucosa and/or overlying mucosa/villous structure. For example, the collected mucosa can be shaved, and the submucosa mechanically dissected. Stem cells can be grown on and/or in a gel-based matrix, such as to generate a solid and/or semi-aqueous product form.

Processing device 500 can be used to amplify stem cells (e.g. intestinal stem cells) outside of the patient, and it can include components to suspend the result in a saline or balanced salt solution.

Processing device 500 can be configured to perform an ex-vivo enriched cell culturing process on tissue 61, such as a process in which a medium that includes trophic factors is used.

Processing device 500 can be configured to perform an encapsulation, such as to include cells in a scaffold or other cell-carrying component.

Processing device 500 can be configured to perform cell sorting, such as to dissociate cells, suspend them in solution, and use a cell sorting mechanism to select stem cells. Stem cells can be selected based on cell surface receptors, and they then can be amplified prior to depositing at one or more deposit sties.

Processing device 500 can be configured to remove collagen from tissue 61, such as to isolate crypts.

Processing device 500 can be configured to cause (e.g. allow) cells (e.g. cells of tissue 61) to grow into a material 60 comprising organoids and/or it may cause the cells to grow into a material 60 comprising a monolayer sheet of cells. The organoids may be dissociated prior to deposition of material 60 and/or they may be maintained as organoids at the time of material 60 deposition.

Processing device 500 can combine tissue 61 with an agent, such as an agent 62 comprising a material selected from the group consisting of: trophic factor; antioxidant; salicylate; a nonsteroidal anti-inflammatory drug (NSAID); and combinations of one, two, or more of these. In some embodiments, agent 62 is added at a time near the time material 60 is deposited in the patient (e.g. within 8 hours of the depositing of material 60 at the deposit site).

Processing device 500 can include one or more scaffolds, such as a scaffold in which tissue is grown (e.g. in combination with cell culturing). The scaffold may comprise a cellular or acellular scaffold. The scaffold may comprise small intestinal tissue from a human or other mammal. The scaffold can comprise small intestinal submucosa, such as porcine small intestinal submucosa. The scaffold can comprise a gelatinous protein mixture configured as a basement membrane matrix (e.g. Matrigel or Cultrex BME). This basement membrane matrix can comprise a shear thinning hydrogel.

Processing device 500 can be configured to arrange cells in a hydrogel matrix, such as when material 60 includes the hydrogel matrix (e.g. the hydrogel matrix is deposited at the deposit site). The hydrogel matrix can be configured to degrade over time in the patient.

Processing device 500 can be configured to modify tissue (e.g. tissue 61 and/or other tissue) in one or more ways, such as to genetically, chemically, and/or epigenetically modify tissue 61.

Processing device 500 can be configured to perform a transgenic treatment of tissue (e.g. a transgenic modification of tissue 61 and/or other tissue), to cause a targeted expression (e.g. an increase or decrease in expression) in the tissue generated by depositing material 60 (e.g. a transgenic transformation causes the generated tissue to hyper-secrete and/or hyposecrete desired hormones). For example, processing device 500 can perform a transgenic treatment to cause the resultant tissue (tissue generated by depositing of material 60) to express exenatide or GLP-1 or a GLP-1 analogue, such as when system 10 is configured to treat obesity, Type 2 diabetes (with or without obesity also being present), non-alcoholic fatty liver disease (NAFLD), non-alcoholic steatohepatitis (NASH), and/or early stage Type 1 diabetes (e.g. in a patient with sufficient residual beta cell mass, such as to maintain and/or increase that mass). In these embodiments, processing device 500 can further cause the deposited material 60 to express PYY and/or oxyntomodulin or Glicentin (to enrich and/or de-enrich the tissue for certain types of enteroendocrine cells, to enhance, suppress, and/or modify the hormonal contents of enteroendocrine secretion), such as when system 10 is configured to treat Type 2 diabetes. In some embodiments, processing device 500 can perform a transgenic treatment to cause the resultant tissue (that is generated based on the depositing of material 60) to express (e.g. start or at least increase the secretion of): GLP-1; PYY; GIP; CCK; glicentin; oxyntomodulin; exenatide; exendin 9-39; ghrelin; CCK antagonists; FGF1; FGF19; FGF21; amylin; insulin; leptin; adiponectin; GLP-2; and/or a peptide that engenders insulin sensitization, insulin resistance, and/or satiety. In some embodiments, a transgenic treatment of tissue is performed on tissue to cause the resultant tissue to express the above hormones in response to luminal contents (e.g. nutrients, bile salts, and the like), and/or express these hormones in response to blood components (e.g. glucose). The deposited material 60 can be configured to express antibodies that inhibit the action of one, two, or any combination of these proteins. Inhibition of select proteins and activation or expression of other proteins can be performed combinatorially. The combinatorial use can be determined by the patient disease state and/or some other measurable physiologic parameter, such as insulin resistant state, diabetes status, blood sugar levels, liver fat, liver fibrosis, fertility, BMI, and/or other factors.

Processing device 500 can perform a transgenic modification that includes Crispr/Cas9 gene editing of the somatic DNA or similar gene editing tools that perform a similar function to CRISPR/Cas9 gene editing. For example, processing device 500 can perform a transgenic modification that includes use of a PiggyBac transposon, lentiviral vector, and/or AAV vector.

Processing device 500 can perform processing that includes decontamination of tissue to alter the microbial content of the tissue (e.g. harvested tissue 61) prior to material 60 being deposited in the patient. This decontamination can be achieved in a variety of ways, such as the incubation of the cells with antibiotics for a specific length of time, such as at least 1 day, 2 days, or 5 days. The process can involve replacing the culture media in which the tissue is growing, such as to a culture media that does not contain antibiotics. Optionally, the processing performed by processing device 500 can involve a confirmatory step to assure that material 60 does not include any infective material, such as a confirmatory step including a test for endotoxins.

Processing device 500 can be configured to eliminate or at least reduce (“reduce” herein) microbes, such as microbes that are included in the harvesting of tissue 61. For example, tissue 61 can be processed in an antibiotic medium, and/or microbes can be strained with a filter (e.g. a filter in which microbes pass through but larger cells, such as stem cells, do not).

Processing device 500 can be configured to perform a “tagging” of tissue, such that material 60 includes a fluorescent or other marker used to identify material 60 (and/or resultant material, also referred to as “material 60” herein) after depositing in the patient. Tagging can be used to assess procedure longevity, to assess the efficacy of the depositing procedure, and/or to identify tissue to be removed in a subsequent procedure if desired (e.g. an undesired effect is encountered and should be reversed or reduced).

Processing device 500 can be configured to add an agent 62 comprising a bioadhesive agent (e.g. a bioadhesive polymer) to material 60, such as an agent configured to cause or enhance adhesion of material 60 to a tissue surface at a deposit site.

In some embodiments, processing device 500 is configured to treat in-situ tissue prior to its harvest. For example, processing device 500 can be inserted into the patient's GI tract, such as to allow an operator to inject one or more agents (e.g. agent 62 and/or 70 described herein) proximate tissue to be harvested (e.g. tissue 61 harvested at least 1 day after the injection).

Material 60 can comprise tissue 61, and/or processed tissue 61 (e.g. tissue 61 that has been modified and/or otherwise processed by processing device 500 as described herein). As used herein, the term “tissue 61” can comprise unprocessed tissue 61 and/or processed tissue 61.

In some embodiments, material 60 can comprise one or more components that are configured to pass through the membrane of one or more cells at a deposit site.

In some embodiments, material 60 does not include tissue 61, such as is described herebelow in reference to method 100″ of FIG. 4. For example, material 60 can comprise a transgenic virus and/or other tissue modifying material that is applied to and/or within a deposit site.

In some embodiments, material 60 comprises one or more agents, such as agent 62 described herein.

In some embodiments, material 60 includes one or more components 63 configured to aid in the depositing of material 60 and/or to maintain material 60 at the deposit site.

For example, component 63 can comprise a deployment device, carrier 63a, such as a stent-like device onto which the other materials of material 60 (e.g. tissue 61) is deposited, the stent-like device deployed within a lumen (e.g. the lumen of the small intestine or other body lumen) at the deposit site location. Carrier 63a can comprise a full or partial circumferential tubular structure, such as a structure seeded with tissue 61. Carrier 63a can comprise a tubular structure comprising a hydrogel.

Alternatively or additionally, component 63 can comprise an adhesive, adhesive 63b, such as an adhesive gel, such as an adhesive gel used to secure material 60 to the deposit site and/or to secure multiple components of material 60 together.

Alternatively or additionally, component 63 can comprise a coating or wrap, coating 63c, such as a coating or wrap configured to prevent material 60 from migrating from the deposit site (e.g. at an undesired time). In some embodiments, component 63 comprises a carrier 63a, and a coating 63c configured as a protective coating or other protecting element. In some embodiments, component 63 comprises a coating 63c that is applied to a deposit site in one or more procedures performed after the depositing of material 60 at the deposit site (depositing of a material 60 with or without coating 63c), as described herebelow in reference to Step 170 of FIGS. 3-4. In these embodiments, carrier 63a protects other components of material 60 (e.g. tissue 61), such as to prevent undesired migration of material 60.

Material 60 can comprise tissue provided en bloc.

Material 60 can comprise biobanked tissue. For example, material 60 can comprise tissue that is treated, and then stored (e.g. frozen), and/or tissue that is stored (e.g. frozen), and subsequently treated.

Agent 62 can comprise one or more pharmaceutical drugs, nutrients (e.g. hexoses, lipids, and/or amino acids), vitamins (e.g. water soluble vitamins such as ascorbic acid), buffering agents, chemicals, fillers, and/or other agents that are included in material 60. In some embodiments, agent 62 comprises one or more agents selected from the group consisting of: antibiotic; adhesive agent such as a bio-adhesive agent; a trophic agent (e.g. a growth factor or other factor used to promote wound healing): a shielding agent (e.g. an agent configured to protect one or more components of material 60 after depositing); and combinations of one, two, or more of these.

In some embodiments, one or more agents 62 are included in a process for creating material 60 at a time that is proximate the time that material 60 is deposited at a deposit site, such as a time within 8 hours of the depositing of material 60 at a deposit site.

In some embodiments, system 10 includes one or more pharmaceutical drugs or other agents, agent 70 that is delivered to the patient prior to the depositing of material 60, and/or after the depositing of material 60, such as is described herebelow in reference to FIGS. 3-4. Agent 70 can be delivered to the patient orally, transdermally, via an injection (e.g. a subcutaneous, intramuscular, epidural, and/or intrathecal injection); and/or intravascularly (e.g. intravenously and/or intraarterially).

In some embodiments, agent 70 comprises one or more: antibiotics; probiotics; and/or prebiotics.

In some embodiments, agent 70 comprises iron, such as when material 60 comprises tissue 61 that has been harvested by harvesting device 400 from the ileum, and depositing device 600 deposits material 60 in the duodenum and/or proximal jejunum. In these embodiments, the iron-based agent 70 can be delivered to the patient to prevent an iron insufficiency otherwise caused by the depositing of material 60.

In some embodiments, agent 70 comprises one or more of: an anti-inflammatory agent; an NSAID, and/or an immunosuppressant.

In some embodiments, depositing device 600 comprises one or more functional elements 699. In some embodiments, system 10 comprises a harvesting device 400 that comprises one or more functional elements 499. In some embodiments, system 10 comprises a processing device 500 that comprises one or more functional elements 599. In some embodiments, system 10 comprises a treatment device 700 that comprises one or more functional elements 799. Functional elements 499, 599, 699, and/or 799 can comprise one, two, or more sensors, transducers, and/or other functional elements as described herein.

Referring now to FIG. 2, a flow chart of a method for depositing material at a deposit site of a patient is illustrated, consistent with the present inventive concepts. Method 100 of FIG. 2 shall be described in reference to the use of system 10 of FIG. 1. In some embodiments, method 100 is configured to treat one, two or more medical conditions of the patient (e.g. a mammalian patient), such as is described herein.

In Step 110, a patient is selected, such as is described herebelow.

In Step 130, tissue 61 is harvested, such as is described herebelow.

In Step 160, a material, such as material 60 described herein, is deposited at one or more deposit sites such as is described herebelow.

Referring now to FIG. 3, a flow chart of another method for depositing material at a deposit site of a patient is illustrated, consistent with the present inventive concepts. Method 100′ of FIG. 3 includes numerous optional steps, and it shall be described in reference to the use of system 10 of FIG. 1. In some embodiments, method 100′ is configured to treat one, two or more medical conditions as described herein.

In Step 110, a patient is selected, such as is described herebelow.

In Step 120, an optional step of performing a pre-harvest procedure, Procedure A, can be performed, such as is described herebelow.

In Step 130, tissue 61 is harvested, such as is described herebelow.

In Step 140, an optional step of processing the harvested tissue 61 can be performed, such as is described herebelow.

In Step 150, an optional step of performing a pre-deposit (and post-harvest) procedure, Procedure B, can be performed, such as is described herebelow.

In Step 160, a material, such as material 60 described herein, is deposited at one or more deposit sites, such as is described herebelow.

In Step 170, an optional step of performing a post-deposit procedure, Procedure C, can be performed, such as is described herebelow

Referring now to FIG. 4, a flow chart of another method for depositing material at a deposit site of a patient is illustrated, consistent with the present inventive concepts. Method 100″ of FIG. 4 includes numerous optional steps, and it shall be described in reference to the use of system 10 of FIG. 1. In some embodiments, method 100″ is configured to treat one, two or more medical conditions as described herein.

In Step 110, a patient is selected, such as is described herebelow.

In Step 140′, an optional step of material processing is performed to produce material 60. For example, material processing can be performed by processing device 500 to create a transgenic virus and/or other tissue modifying material, material 60, that is applied to and/or within a deposit site. Depositing of this material 60 can cause resultant tissue to be generated that expresses one or more proteins which provide one or more therapeutic benefits to the patient, as described herein.

In Step 150, an optional step of performing a pre-deposit procedure, Procedure B, can be performed, such as is described herebelow.

In Step 160, material 60 is deposited at one or more deposit sites, such as is described herebelow.

In Step 170, an optional step of performing a post-deposit procedure, Procedure C, can be performed, such as is described herebelow.

The details provided herebelow describe the steps, including the optional steps, of methods 100, 100′, and/or 100″ of FIGS. 2, 3, and/or 4, respectively.

In some embodiments, Step 110 of methods 100, 100′, and/or 100″ (singly or collectively method 100 herein), selects a patient (e.g. a mammalian patient) with an HbA1c level of at least 6.5%, at least 7.0%, at least 7.5%, or at least 8%. In some embodiments, in Step 110 of method 100, a patient is selected that is obese, has a body mass index (BMI) of at least 30, has Type 2 diabetes, and has a HbA1c level above 7.0.

In some embodiments, a patient is selected in Step 110 that has ghrelin levels that exceed a threshold, such as when ghrelin levels are at or above 20 μg/ml. In some embodiments, a patient is selected that has leptin levels that exceed a threshold (e.g. fall below a minimum threshold), such as when leptin levels are at or below 100 fmol/l.

In some embodiments, a patient is selected that has a BMI of at least 25, 30, 35, and/or 40.

In some embodiments, a patient is selected in Step 110 that has a pancreas with a minimum level of functionality, such as when the patient has a C-Peptide level of at least 0.2, 0.5, 1.0, and/or 2.0 (nM or nmol/l) and/or a fasting inulin of at least 7 μU/ml, 9 μU/ml, or 12 μU/ml.

In some embodiments, a patient is selected in Step 110 that has a relatively non-cirrhotic liver, such as when the patient has: a Fib-4 level of no more than 4; a Fib-4 level of at least 1.3, 2.0, and/or 2.5 (e.g. when the patient has NAFLD and/or NASH); an ALT level of at least 30, 35, and/or 40; an AST level of at least 25, 30, and/or 35; and combinations of one, two, or more of these.

In some embodiments, a female patient is selected in Step 110 that has impaired ovarian function, such as a patient that has oligomenorrhea (e.g. menses with a cycle of at least 35 days) and/or amenorrhea (e.g. no menses for at least 6 months). In some embodiments, a male patient is selected in Step 110 that has impaired testosterone levels and/or impaired sexual function (such as impotence).

In some embodiments, a patient is selected in Step 110 that has lipodystrophy, such as when the patient has: a triglyceride level of at least 150 mg/dl and/or 200 ml/dl; an HbA1c level of at least 6.0%; a low leptin state such as a leptin level of no more than 10.0 μg/ml or 5 μg/ml; and/or genetic markers of lipodystrophy.

In some embodiments, a patient is selected in Step 110 that has insulin resistance, such as when the patient has: a triglyceride/HDL ratio of at least 1.65 and/or 2.75; a HOMA-IR level of at least 2.0, 2.5, and/or 3.0; a fasting insulin of at least 5 μU/ml, 7 μU/ml, and/or 10 μU/ml; and/or a fasting glucose of at least 100 mg/dl and/or 125 mg/l.

In some embodiments, a patient is selected in Step 110 that has liver disease, such as fatty liver disease or other liver disease diagnosed and/or prognosed via magnetic resonance (MR) imaging and/or ultrasound imaging. For example, magnetic resonance imaging can be used to determine a proton densitometry fat fraction (PDFF), also referred to as magnetic resonance fat fraction, where the patient is selected for treatment if their intra-hepatic triglyceride (IHTG) is at least 5%. Ultrasound imaging can be used to determine a hepato-renal index of echo levels (HRI), wherein the patient is selected for treatment if the HRI difference is at least 4.0 dB and/or the HRI ratio is no more than 1.0. A fibroscan (transient elastography) can be used to assess a patient for treatment. For example, a patient can be selected for treatment if their vibration controlled transient elastography is determined to be at least 6 kPa, or at least 8 kPa (e.g. when measured at 50 Hz). Alternatively or additionally, a patient can be selected for treatment if their controlled attenuation parameter (CAP), also referred to as ultrasound attenuation rate, is at least 200 dB/m or at least 250 dB/m.

In some embodiments, a patient is selected in Step 110 that has a metabolic disease, such as a metabolic disease diagnosed and/or prognosed via metabolomics. For example, a patient can be selected for treatment if their level of 2-hydroxybutyrate is no more than 5 μg/ml.

In some embodiments, a patient is selected in Step 110 that has an iron storage disorder. An example of an iron storage disorder may be iron overload, such as that associated with metabolic syndrome, type 2 diabetes, insulin resistance, fatty liver disease, and/or steatohepatitis. Alternatively or additionally, an example of an iron storage disorder may be hereditary hemochromatosis. For example, a patient can be selected for treatment if their ferritin level is determined by system 10 to be at least 50 ng/ml, or at least any of 60 ng/ml, 80 ng/ml, 100 ng/ml, 200 ng/ml, 250 ng/ml, and/or 500 ng/ml.

In some embodiments, a patient is selected in Step 110 that has celiac disease and/or gluten insensitivity. For example, a patient can be selected if a duodenal biopsy shows villous atrophy.

In some embodiments, a patient is selected in Step 110 that has short bowel syndrome, such as when treatment (e.g. ablation) of the intestinal mucosa and/or implantation of material 60 in the intestinal mucosa alleviates symptoms or otherwise treats a patient's short bowel syndrome.

In some embodiments, a patient is selected in Step 110 that has post-bariatric hypoglycemia. For example, a post-bariatric surgery patient can be selected: if a post-prandial blood sugar level is determined by system 10 to be below 70 mg/dl, 60 mg/dl, and/or 50 mg/dl; if the patient has known hypoglycemia unawareness; and/or if the patient has documented history of severe hypoglycemia, such as one or more hypoglycemic events requiring third party assistance.

In some embodiments, a patient is selected in Step 110 that has a genetic predisposition, such as a predisposition toward lipodystrophy, a lipid disorder, an iron storage disorder, and/or monogenic diabetes. In these embodiments, a genetic test may be performed in Step 120 to screen the patient.

In some embodiments, a patient is selected in Step 110 that has a hormonal defect, such as a patient with insufficient GLP-1 secretions and/or PYY secretions (e.g. as determined by measuring circulating levels of these).

Method 100′ includes optional Step 120 in which a procedure is performed, Procedure A, a procedure performed prior to harvesting of tissue in Step 130. Procedure A can be performed on one or more mammals from which the tissue is to be harvested, which can include the patient and/or another mammal. In some embodiments, Procedure A is performed on the patient and or another mammal (patient and/or other mammal “patient” herein), such as when tissue is harvested from both the patient and another mammal.

Procedure A of Step 120 can comprise performing a diagnostic procedure on the patient, such as to determine if the remaining steps of the procedure should be performed (e.g. determine if the tissue 61 harvesting of Step 130 and/or the material 60 depositing of Step 160 should be performed). For example, a diagnostic procedure can be performed to measure serum ferritin, such as when the procedure is continued if serum ferritin is at least 200 ng/ml, at least 250 ng/ml, and/or at least 500 ng/ml. In some embodiments, the patient is assessed for mucosal hyperplasia (e.g. via OCT, chromoendoscopy, and/or other endoscopy), and the procedure is continued if mucosal hyperplasia is at or above an acceptable level. In some embodiments, the patient is assessed for the presence of ulcers, diverticula, and/or cysts (e.g. via an endoscopic or other visualization procedure), and the procedure is continued if these issues are not present or at or above an acceptable level.

Procedure A of Step 120 can comprise delivery of one or more agents to the patient, such as agent 70 described hereabove in reference to FIG. 1 (e.g. agents delivered to the patient that is receiving material 60 and/or to another mammal from which tissue 61 is to be harvested). For example, procedure A of Step 120 can comprise a microbiome adjusting procedure to be performed on the patient. In some embodiments, agent 70 comprises one or more antibiotics, probiotics, and/or prebiotics that can be given to the patient (e.g. when accompanied by a “nurturing” procedure after the material 60 depositing performed in Step 160).

Procedure A of Step 120 can comprise a pharmaceutical or other agent regimen undertaken by the patient (e.g. agents delivered to the patient that is receiving material 60 and/or to another mammal from which tissue 61 is to be harvested). For example, the patient may take an agent selected from the group consisting of: metformin; GLP-2; a probiotic; a prebiotic; and combinations of one, two, or more of these. In some embodiments, one or more of the agents listed above are used to alter the properties of the mucosal tissue at the harvest site.

Procedure A of Step 120 can include one or more surgical or other procedures performed on the patient (e.g. procedures performed on the patient that is receiving material 60 and/or to another mammal from which tissue 61 is to be harvested). For example, a mucosal treatment procedure, such as a mucosal ablation or mucosal removal procedure performed using treatment device 700, can be performed in Step 120, such as proximate (e.g. at and/or near) the harvest location of Step 130 and/or the deposit location of Step 160. In some embodiments, an agent delivery procedure is performed, such as when one or more agents 70 are injected into tissue proximate the harvest location of Step 130 and/or the deposit location of Step 160.

Procedure A of Step 120 can comprise a diet undertaken by the patient (e.g. a diet undertaken by the patient that is receiving material 60 and/or to another mammal from which tissue 61 is to be harvested), such as a diet selected from the group consisting of: a high sugar diet; a low sugar diet; a high carbohydrate diet; a low carbohydrate diet; a high fiber diet; a low fiber diet; a fast carbohydrate diet; a slow carbohydrate diet; a liquid diet; and combinations of one, two, or more of these. In some embodiments, one or more of the diets listed above is performed to alter mucosal thickness in the harvest site. In some embodiments, application of a hexose-based agent (e.g. fructose or glucose-based agent) can be applied to the harvest site (e.g. a mouthwash applied to the mouth) to cause hyperplastic tissue growth at the harvest site.

In some embodiments, procedure A is performed at least 1 week before the harvesting of tissue 61 of Step 130, such as when procedure A comprises a diet for the patient.

Step 130 of methods 100, 100′, and 100″ includes harvesting tissue 61. Tissue 61 is harvested at one or more harvesting sites, such as one or more harvesting sites of the patient and/or another mammal. In some embodiments, tissue 61 comprises tissue of the patient that is harvested, and tissue of another mammal that is harvested (either or both referred to as “patient” herein). In some embodiments, the harvesting of tissue 61 in Step 130 is performed using harvesting device 400 and/or otherwise as is described hereabove in reference to FIG. 1.

In some embodiments, Step 130 includes avoiding harvesting tissue of: the lower esophageal sphincter; the pylorus; the ampulla of Vater; the ileocecal valve; and combinations of one, two, or more of these.

In some embodiments, Step 130 is performed in multiple clinical procedures, such as multiple clinical procedures performed on different days. In some embodiments, a second harvesting Step 130 is performed during a procedure B of Step 150 or during a depositing procedure of Step 160 (e.g. to harvest tissue to be deposited in a subsequent depositing procedure).

In some embodiments, material 60 comprises tissue 61 that is deposited without processing, such as when material 60 is deposited in the same clinical procedure (e.g. on the same day) as the procedure including the harvesting of tissue 61. Alternatively, at least a portion of material 60 comprises tissue 61 that has been processed in Step 140. In some embodiments, the processing of tissue 61 in Step 140 is performed using processing device 500 and/or otherwise as is described hereabove in reference to FIG. 1.

In some embodiments, material 60 comprises multiple different materials, such as at least a first material 60a, and a second material 60b that is different (e.g. one or more different properties) than first material 60a. In these embodiments, in Step 160, first material 60a can be deposited at a first deposit site, and second material 60b can be deposited at a second deposit site. For example, a first material 60a can include tissue 61a harvested from a distal gut location such as the terminal ileum that is deposited (processed or unprocessed) in the duodenum and/or proximal jejunum, and a second material 60b can include tissue 61b harvested from the duodenum and/or proximal jejunum that is deposited in the terminal ileum.

In some embodiments, the processing performed in Step 150 comprises including in (e.g. adding to) material 60 one or more components configured to aid in the depositing of material 60 and/or to maintain material 60 at the deposit site, such as is described hereabove in reference to FIG. 1.

In some embodiments, the processing performed in Step 150 includes a transgenic treatment of tissue (e.g. tissue 61), such as to cause a targeted expression by material 60 (e.g. a transgenic transformation causes material 60 (and/or resultant material, also referred to as “material 60” herein) to hyper-secrete desired proteins, such as hormones), such as is described herein.

In some embodiments, procedure B of Step 150 comprises a procedure performed using treatment device 700 described hereabove in reference to FIG. 1. For example, procedure B can include a tissue treatment procedure performed proximate (e.g. at and/or near) one or more intended deposit sites of material 60. The tissue treatment procedure can comprise a tissue modification procedure as defined hereabove. For example, tissue can be removed, such that the resultant tissue is “driven by” the characteristics of material 60 deposited in Step 160. In some embodiments, treatment device 700 performs a tissue modification procedure that removes tissue. In these embodiments, treatment device 700 can use thermal ablation (e.g. heat and/or cryo-based ablation), energy-based ablation, chemical ablation, and/or mechanical extraction, to remove the tissue (e.g. mucosal tissue) proximate (e.g. at and/or near) the intended deposit site for material 60. A tissue modification procedure can be performed proximal and or distal to the deposit site, such as is described herein. In some embodiments, the tissue modification procedure performed in Step 150 is performed in the same clinical procedure (e.g. on the same day) as the depositing of material 60 performed in Step 160. Alternatively, the tissue modification procedure performed in Step 150 can be performed at least 1 day, at least 2 days, at least 3 days, at least 1 week, and/or at least 2 weeks prior to the depositing of material 60 in Step 160 (e.g. to allow the treated tissue to slough off prior to the depositing of material 60).

In some embodiments, a first step of procedure B includes delivery of energy and/or an agent to tissue (e.g. by a treatment device 700 to cause the tissue to necrose), and a second step includes removing the treated tissue (e.g. by the same or a different treatment device 700, in the same or a subsequent clinical procedure). In these embodiments, tissue can be removed proximate one or more deposit sites at which material 60 is to be deposited (e.g. deposited using depositing device 600).

Procedure B of Step 150 can comprise a diet undertaken by the patient, such as a diet selected from the group consisting of: a high sugar diet; a low sugar diet; a high carbohydrate diet; a low carbohydrate diet; a high fiber diet; a low fiber diet; a fast carbohydrate diet; a slow carbohydrate diet; a liquid diet; and combinations of one, two, or more of these. In some embodiments, procedure B comprises a diet configured to change the mucosal thickness at the intended deposit site.

Procedure B of Step 150 can comprise a diagnostic procedure, such as an endoscopic or other visualization procedure used to assess one or more deposit sites of the patient.

Procedure B of Step 150 can be performed in the same clinical procedure as the harvesting of tissue 61 of Step 130, at a time before Step 130 (e.g. at least 8 hours before), and/or at a time after Step 130 (e.g. at least 8 hours). Procedure B can be performed at a minimum time before the depositing of material 60 performed in Step 160. Alternatively or additionally, Procedure B of Step 150 can be performed within a maximum time of the performance of Step 160. In some embodiments, Procedure B comprises a mucosal or other tissue modification procedure, and the depositing of material 60 performed in Step 160 is performed at least 1 day, or at least 2 days after Procedure B. Alternatively or additionally, procedure B can comprise a mucosal or other tissue modification procedure that is performed no more than 7 days before the depositing of material 60 of Step 160.

In some embodiments, material 60 is deposited in Step 160 at one, two or more deposit sites, such as by using depositing device 600 described hereabove in reference to FIG. 1. Material 60 can be deposited at the one, two or more deposit sites to treat one or more medical conditions of the patient, as described herein. In some embodiments, material 60 comprises multiple different materials, such as at least a first material 60a, and a second material 60b, as described herein. In these embodiments, first material 60a can be deposited at a first deposit site, and second material 60b can be deposited at a second deposit site, also as described herein.

In some embodiments, material 60 is deposited at multiple, relatively small, neighboring locations, such as to allow mucosal tissue growth in-vivo (e.g. each deposit location is a nidus for resultant tissue generation). For example, material 60 can be deposited at multiple sites of no more than 10 mm² in size.

In some embodiments, material 60 is deposited into one or more deposit sites comprising submucosal tissue (e.g. submucosal tissue of the intestine), after a tissue modification procedure has been performed (in Step 150) of the mucosal tissue proximate the one or more deposit sites. For example, material 60 can be injected and/or otherwise deposited at the submucosal target site at least 1 day, 2 days or 3 days after the mucosal tissue modification procedure has been performed. In some embodiments, material 60 is deposited at the submucosal target site within 7 days, within 5 days, and/or within 1 day of the mucosal tissue modification procedure.

In some embodiments, material 60 includes a component configured to aid in the depositing of material 60 and/or to maintain material 60 at the deposit site, such as is described hereabove in reference to FIG. 1.

As described hereabove, a tissue modification procedure (e.g. a mucosal modification procedure) can be performed (e.g. in Step 150 using treatment device 700) at a deposit site, prior to the depositing of material 60 in Step 160. In these embodiments, material 60 can be deposited along the margins of the treated tissue area, and material 60 can “grow in” from the margins. Alternatively or additionally, material 60 can be deposited within the margins of the treated tissue area.

In some embodiments, material 60 is deposited in a deposit site that comprises one or more axial lengths of intestinal or other GI segments with a single or cumulative length of at least 1 cm, at least 3 cm, at least 10 cm, or at least 20 cm. As described hereabove, in some embodiments, material 60 is deposited within a cumulative surface area (e.g. surface area of the inner wall of one or more segments of the GI tract) of at least 50 cm², at least 100 cm², or at least 250 cm² (e.g. material is deposited into one or more “patches” that cover 1% to 100% of that surface area). In some embodiments, material 60 is deposited at a deposit site that comprises a relatively full circumferential portion of a body lumen such as an axial segment of the duodenum, jejunum, and/or other GI conduit. Alternatively or additionally, material 60 can be deposited along a partial circumferential portion (e.g. less than 240°, less than 180°, and/or less than 90° of the circumference) of a GI and/or other body lumen. Material 60 can be deposited along a partial circumferential portion of at least 45°, or at least 90° of the circumference. As described herein, material 60 can be deposited at a single or multiple deposit sites, such as at least 10 deposit sites, at least 20 deposit sites, or at least 1000 deposit sites.

In some embodiments, the depositing of material 60 of Step 160 is performed in the same clinical procedure (e.g. within 8 hours) of the harvesting of tissue 61 of Step 130. Alternatively or additionally, the depositing of material 60 can be performed in a different, second clinical procedure, such as a procedure performed at least 1 day, and/or at least 2 days after the harvesting of tissue 61 performed in Step 130. The second procedure can be performed within a time limit, such a time limit of no more than 5 days of the time of harvesting performed in Step 130.

In some embodiments, procedure C of Step 170 is performed using treatment device 700 described hereabove in reference to FIG. 1. For example, a tissue modification procedure can be performed at a location proximal or distal to the deposit site, such as to allow migration of material 60 and/or resultant tissue to the treated area.

Procedure C of Step 170 can comprise a diet undertaken by the patient, such as a diet selected from the group consisting of: a high sugar diet; a low sugar diet; a high carbohydrate diet; a low carbohydrate diet; a high fiber diet; a low fiber diet; a fast carbohydrate diet; a slow carbohydrate diet; a liquid diet; a soft-foot diet; and combinations of one, two, or more of these. In some embodiments, procedure A of Step 120 and/or procedure B of Step 150 can also comprise the patient undergoing a diet, and the diet of Procedure C can be a different diet than that of procedure A and/or procedure B. For example, in some embodiments, procedure A comprises a high carbohydrate diet (e.g. a diet of at least 7 days) that is configured to stimulate local mucosa hyperplasia (prior to harvesting of tissue 61). Subsequently, procedure C comprises a liquid diet (e.g. of at least 7 days) that is followed by a low carbohydrate diet (e.g. of at least 28 days), the two diets performed sequentially after the depositing of material 60.

In some embodiments, the elapsed time between Step 160 and Procedure C is at least a minimum duration of time (for example, at least one month, 3 months, 6 months, and/or 12 months). In some embodiments, the elapsed time between Step 160 and Procedure C is no more than a maximum duration of time (for example, no more than one month, 3 months, 6 months, and/or 12 months).

Procedure C of Step 170 can comprise delivery of one or more agents to the patient, such as agent 70 described hereabove in reference to FIG. 1.

In some embodiments, procedure C comprises delivery of an agent 70 that comprises iron, such as when tissue 61 comprises tissue of the terminal ileum and the deposit site is within the duodenum and/or proximal jejunum.

In some embodiments, procedure C comprises delivery of an agent 70 that comprises a microbiome companion, such as an agent used to promote proper healing (e.g. which can be combined with a manipulation of the microbiome in Procedure A of Step 120).

In some embodiments, procedure C comprises delivery of an agent 70 that comprises an NSAID and/or an immunosuppressant (e.g. when tissue 61 comprises an allograft and/or a xenograft).

In some embodiments, procedure C comprises delivery of nurturing agents, such as nutrients (hexoses, lipids, amino acids), vitamins (e.g. water-soluble vitamins such as ascorbic acid), and/or buffering agents.

In some embodiments, material 60 comprises coating 63c described hereabove in reference to FIG. 1. Procedure C can include applying coating 63c at a deposit site, such as a coating applied to protect other components of material 60 (e.g. tissue 61), such as to prevent undesired migration of material 60. In these embodiments, procedure C can include applying component 63c multiple times, such as in multiple steps 170 that are performed routinely over a duration of up to 1 week, up to 4 weeks, and/or up to 12 weeks (e.g. a procedure performed repeatedly every 1 through 14 days).

In some embodiments, procedure C comprises a diagnostic procedure, such as an endoscopic or other visualization procedure.

In some embodiments, procedure C comprises a “reversal procedure” configured to reverse or at least reduce the effects on the patient of the depositing of material 60 (and/or other effects caused by the previous steps of methods 100, 100′, and/or method 100″). For example, a tissue sample can be taken (e.g. using harvesting device 400) at one or more deposit sites, prior to the implantation of material 60 at the deposit site(s). The deposit site tissue can be stored (e.g. in a freezer). The stored material can be modified as described in step 140 before and/or after being stored. If an undesired result is obtained after depositing of material 60 at the one or more deposit sites, the stored tissue can be deposited (e.g. using depositing device 600) at the deposit site to cause the reversal (e.g. after the deposit site tissue is removed or at least modified, such as via an ablation or other tissue modifying procedure performed using treatment device 700). The deposit performed in Procedure C can include depositing material over the entirety of the earlier deposit site, over a partial segment of the earlier deposit site, and/or over a larger area than the earlier deposit site.

In some embodiments, procedure C comprises an “augmentation procedure” configured to increase the effects on the patient of the depositing of material 60 (and/or other effects caused by the previous steps of methods 100, 100′, and/or method 100″). If a desired result is obtained after depositing of material 60 at the one or more deposit sites, but a greater magnitude or duration of the effect is also desired, additional material 60 (e.g. material that had been stored) can be deposited (e.g. using depositing device 600) proximate the deposit site to cause the augmentation (e.g. after the deposit site tissue is removed or at least modified, such as via an ablation or other tissue modifying procedure performed using treatment device 700). The deposit performed in Procedure C can include depositing material over the entirety of the earlier deposit site, over a partial segment of the earlier deposit site, and/or over a different area than the earlier deposit site.

In some embodiments, Procedure C can include a reversal and/or augmentation procedure performed to achieve multiple effects. As an example, Procedure C may be configured to augment weight loss in a patient while reversing nausea.

In some embodiments, system 10 and the methods of the present inventive concepts are configured to treat one, two or more medical conditions as described hereabove in reference to FIG. 1.

System 10 and the methods of the present inventive concepts can be configured to treat at least two medical conditions, such as Type 2 diabetes and NAFLD, Type 2 diabetes and hypertension; Type 2 diabetes and hypertriglyceridemia; Type 2 diabetes and iron overload; NALFD and iron overload; NASH and iron overload; or Type 2 diabetes and NASH. In these embodiments, harvesting device 400 can be used to harvest tissue 61 from one or more tissue locations, such as one or more locations that do not include duodenal tissue and/or proximal jejunal tissue (e.g. not tissue from the proximal 50 cm of jejunum). For example, tissue 61 can comprise tissue of the ileum and/or colon. Alternatively in these embodiments, tissue 61 can comprise tissue of the duodenum or proximal jejunum that is modified by processing device 500. For example, processing device 500 can modify tissue 61 to cause it to express proteins (e.g. hormones) that are normally expressed in the ileum, such as GLP-1 or PYY. After harvest, material 60 (e.g. material 60 comprising tissue 61, modified tissue 61, and/or other material, each as described herein) can be deposited at a deposit site, such as a deposit site including the duodenum and/or the proximal jejunum (e.g. the proximal 50 cm of jejunum). Also in these embodiments, treatment device 700 can be used to treat tissue proximate the deposit site (e.g. tissue of the deposit site and/or tissue near the deposit site), such as an ablation or other treatment procedure that is performed prior to the depositing of material 60.

In some embodiments, in Step 130, tissue 61 is harvested by harvesting device 400 from a first intestinal location, and in Step 160, material 60 is deposited by depositing device 600 at a second intestinal location that is different than the first intestinal location. In these embodiments, material 60 can comprise processed or unprocessed tissue 61 (e.g. processing by processing device 500 as described herein). For example, tissue 61 can comprise tissue harvested from the colon which is then deposited (via inclusion of tissue 61 in the deposited material 60) in the duodenum and/or proximal jejunum. The resultant tissue generated proximate the deposit site will exhibit the properties of tissues of the colon.

In some embodiments, further screening procedures (such as diagnostic endoscopy procedures) can be performed, such as to screen for the risk of new incident pathology in the deposited location (e.g. a screening performed at regular intervals, such as every 3 years, every 5 years, or every 10 years).

In some embodiments, in Step 130, tissue 61 is harvested by harvesting device 400 from a first intestinal location, and in Step 160, material 60 is deposited at the same intestinal location. In these embodiments, material 60 can include tissue 61 that is modified in Step 140, such as when tissue 61 is genetically, chemically, and/or epigenetically modified (e.g. a modification performed by processing device 500 as described herein). For example, tissue 61 can comprise tissue harvested from the duodenum and/or proximal jejunum which is then modified (e.g. a cellular function modification performed by processing device 500 as described herein), and subsequently placed (via including of the modified tissue 61 in the deposited material 60) in the duodenum and/or proximal jejunum (e.g. in the same or a similar location as the harvest site). The resultant tissue generated proximate the deposit site will exhibit the properties of the modified tissue. For example, tissue 61 may be harvested by harvesting device 400 in a patient with celiac disease. The tissue 61 can be genetically modified by processing device 500 to express a substance (e.g. a protein and/or RNA aptamer) that blocks the immunogenicity of the antigen (such as by expressing an enzyme that can degrade gluten in ingested food). Material 60 can include this modified tissue 61, and subsequently it can be deposited into the duodenum of the patient. In another example, tissue 61 may be harvested by harvesting device 400 in a patient with hereditary hemochromatosis. The tissue 61 may be genetically modified to express a protein that expresses a functional HFE gene in a manner that can restore normal HFE protein function in the modified tissue. Material 60 comprising this modified tissue 61 can be subsequently deposited into the duodenum of the patient.

In some embodiments, in Step 130, tissue 61 comprises tissue of the terminal ileum and/or colon that is harvested by harvesting device 400 (e.g. tissue that include mucosal tissue including crypts and/or stem cells). In these embodiments, material 60 (e.g. material 60 comprising tissue 61) can be deposited in the duodenum and/or proximal jejunum (e.g. proximal 50 cm of jejunum) by depositing device 600. The resultant tissue will exhibit the functions and other properties of the distal gut (e.g. the terminal ileum and/or colon), which can recapitulate bariatric surgery-like effects. Alternatively or additionally in these embodiments, material 60 can be deposited at a location selected from the group consisting of: on the skin; subcutaneous tissue; peritoneal cavity; and combinations of one, two, or more of these.

In some embodiments, in Step 130, tissue 61 comprises tissue of the mouth that is harvested by harvesting device 400 (e.g. tissue that include mucosal tissue including crypts and/or stem cells). In these embodiments, material 60 (e.g. material 60 comprising tissue 61) is deposited in the duodenum and/or proximal jejunum (e.g. proximal 50 cm of jejunum) by depositing device 600. The resultant tissue will exhibit entero-endocrine cell function, and this function will now manifest in a more metabolically critical gut site (duodenum and/or proximal jejunum) thus recapitulating bariatric surgery like effects.

In some embodiments, in Step 130, tissue 61 comprises tissue of the esophagus that is harvested by harvesting device 400. In these embodiments, material 60 (e.g. material 60 comprising tissue 61) can be deposited (e.g. by depositing device 600) at a deposit site comprising a metabolically critical gut site, such as the duodenum and/or proximal jejunum (e.g. proximal 50 cm of jejunum). The resultant tissue can exhibit a reduced metabolic function effect (e.g. the resultant tissue does not express proteins that regulate glucose), such as to treat a post-bariatric surgery (e.g. post-RYGB) complication, such as hypoglycemia unintentionally caused by the surgery.

In some embodiments, in Step 130, tissue 61 comprises tissue of the stomach, such as the fundus, antrum, body, and/or cardia of the stomach, that is harvested by harvesting device 400. In these embodiments, material 60 (e.g. material 60 comprising tissue 61) can be deposited (e.g. by depositing device 600) at a deposit site comprising a metabolically critical gut site, such as the duodenum and/or proximal jejunum (e.g. proximal 50 cm of jejunum). The resultant tissue that is generated can exhibit gastric enterocyte secretory function (such as ghrelin secretion) which can be used to treat anorectic and/or cachectic conditions (e.g. underfeeding and/or malnutrition).

In some embodiments, in Step 160, material 60 is deposited in the esophagus by depositing device 600, such as when tissue 61 comprises tissue harvested from healthy esophageal sites, such as to treat Barrett's esophagus and/or eosinophilic esophagitis.

In some embodiments, in Step 160, material 60 is deposited in the stomach by depositing device 600, such as when tissue 61 comprises transgenically modified gastric mucosa tissue (modified to secrete ghrelin), to treat anorectic and/or cachectic conditions (e.g. underfeeding and/or malnutrition).

In some embodiments, in Step 160, material 60 is deposited in the mouth by depositing device 600, such as when tissue 61 comprises transgenically modified distal gut tissue (terminal ileum, colon, and/or other tissue modified to secrete GLP-1), to treat diabetes, obesity, and/or fatty liver disease.

In some embodiments, in Step 160, material 60 is deposited in the base of tongue by depositing device 600, such as when tissue 61 comprises transgenically modified distal gut tissue (terminal ileum, colon, and/or other tissue modified to secrete GLP-1), to treat diabetes, obesity, and/or fatty liver disease.

In some embodiments, method 100 is performed to treat at least Type 2 diabetes and/or obesity, such as to treat both Type 2 diabetes and obesity. In Step 100, a patient is selected, such as a patient with two, three, or more of the following characteristics: obesity; Type 2 diabetes; body mass index of at least 30, and/or an HbA1c of at least 7. In Step 130, tissue 61 is harvested from the colon and/or the terminal ileum, such as by harvesting tissue that include crypts and/or stem cells. In Step 140, tissue 61 is processed to create material 60, such as a processing that includes one, two, or more of: removing collagen (e.g. to isolate crypts); growth of tissue 61 in a culture (e.g. a medium including trophic factors); and/or addition of agent 62 (e.g. an agent including an antioxidant, salicylate, and/or an NSAID that is added at a time proximate the depositing performed in Step 160). In Step 150, a procedure is performed prior to depositing of material 60, procedure B described herein, such as a mucosal tissue modification procedure (e.g. an ablation, denaturing and/or removal procedure). The mucosal tissue modification procedure is performed at or proximate the intended deposit site(s), and can comprise a tissue modification of a full or partial circumferential portion of an axial segment of a GI lumen (e.g. a duodenal or other intestinal lumen), such as a modification of no more than 180° of the circumference of the segment. The mucosal tissue modification procedure can be performed in one or more axial locations, such as to modify a single or cumulative axial length of at least 25 mm. In Step 160, material 60 is deposited in one or more deposit sites in the duodenum and/or proximal jejunum of the patient. The depositing can occur in the same clinical procedure in which procedure B is performed, or in a subsequent clinical procedure performed on a later day. Material 60 can be deposited in the relative geometric middle of the area(s) treated in procedure B, and/or at edge(s) of the treatment area. Material 60 can be deposited in submucosal tissue of the intestine (e.g. in duodenal submucosa via a depositing device 600 comprising one or more needles and/or fluid jets). Step 170 can be performed, comprising procedure C. Procedure C can include one, two, or more of: restricted food intake for a minimum time period (e.g. an elemental diet for approximately 48 hours); a pureed diet for a minimum time period (e.g. at least 2 weeks); and/or intake of one or more NSAID.

System 10 and the methods of the present inventive concepts are configured to treat an adverse event of the patient, such as hypoglycemia (e.g. severe hypoglycemia) that results after the performance of a bariatric procedure (such as an RYGB procedure). In some embodiments, an RYGB treated patient can be selected for treatment that has a post prandial glucose level of no more than 75 mg/dl and/or 50 mg/dl (e.g. as confirmed in a screening procedure performed in Step 120). In these embodiments, in Step 130, harvesting device 400 can be used to harvest tissue 61 from one or more tissue locations, such as a duodenal tissue location (e.g. harvesting of duodenal mucosa that includes crypts and/or stem cells). In Step 140, tissue 61 is processed to create material 60, such as a processing that includes one, two, or more of: removing collagen (e.g. to isolate crypts); growth of tissue 61 in a culture (e.g. a medium including trophic factors); and/or addition of agent 62 (e.g. an agent including an antioxidant, salicylate, and/or an NSAID that is added at a time proximate the depositing performed in Step 160). In Step 150, a procedure can be performed prior to depositing of material 60, procedure B described herein, such as a mucosal tissue modification procedure (e.g. an ablation, denaturing and/or removal procedure). The mucosal tissue modification procedure is performed at or proximate the intended deposit site(s), and the procedure can comprise a tissue modification of a full or partial circumferential portion of an axial segment of a GI lumen, such as a modification of no more than 180° of the circumference of the segment. The mucosal tissue modification procedure can be performed in one or more axial locations, such as to modify a single or cumulative axial length of at least 25 mm. In Step 160, material 60 is deposited in one or more deposit sites in the jejunum of the patient (e.g. in the Roux limb). The depositing can occur in the same clinical procedure in which procedure B is performed, or in a subsequent clinical procedure performed on a later day. Material 60 can be deposited in the relative geometric middle of the area(s) treated in procedure B, and/or at edge(s) of the treatment area. Material 60 can be deposited in submucosal tissue of the intestine (e.g. in jejunal submucosa via a depositing device 600 comprising one or more needles and/or fluid jets). Step 170 can be performed, comprising procedure C. Procedure C can include one, two, or more of: restricted food intake for a minimum time period (e.g. an elemental diet for approximately 48 hours); a pureed diet for a minimum time period (e.g. at least 2 weeks); and/or intake of one or more NSAIDs.

In some embodiments, system 10 and the methods of the present inventive concepts are configured to treat an undesired event that results after a bariatric procedure (e.g. RYGB procedure, sleeve gastrectomy placement, a partial jejunal diversion procedure, and/or a duodenal sleeve placement), such as when the procedure does not sufficiently treat diabetes of the patient and/or results in post-bariatric hypoglycemia. In these embodiments, in Step 130, harvesting device 400 can be used to harvest tissue 61 from one or more tissue locations, such as one or more locations that do not include duodenal tissue and/or proximal jejunal tissue (e.g. not tissue from the proximal 50 cm of jejunum). For example, tissue 61 can comprise tissue of the ileum and/or colon. Alternatively in these embodiments, tissue 61 can comprise tissue harvested from the duodenum and/or proximal jejunum that is modified (e.g. transgenically modified) in Step 140, such as a transgenic modification that causes the resultant tissue to express GLP-1 and/or PYY, as described herein. In Step 140, tissue 61 is processed to create material 60, such as a processing that includes one, two, or more of: removing collagen (e.g. to isolate crypts); growth of tissue 61 in a culture (e.g. a medium including trophic factors); and/or addition of agent 62 (e.g. an agent including an antioxidant, salicylate, and/or an NSAID that is added at a time proximate the depositing performed in Step 160). In Step 150, a procedure can be performed prior to depositing of material 60, procedure B described herein, such as a mucosal tissue modification procedure (e.g. an ablation, denaturing and/or removal procedure). The mucosal tissue modification procedure is performed at or proximate the intended deposit site(s), and it can comprise a tissue modification of a full or partial circumferential portion of an axial segment of a GI lumen, such as a modification of no more than 180° of the circumference of the segment. The mucosal tissue modification procedure can be performed in one or more axial locations, such as to modify a single or cumulative axial length of at least 25 mm. In Step 160, material 60 is deposited in one or more deposit sites in the duodenum and/or proximal jejunum of the patient. The depositing can occur in the same clinical procedure in which procedure B is performed, or in a subsequent clinical procedure performed on a later day. Material 60 can be deposited in the relative geometric middle of the area(s) treated in procedure B, and/or at edge(s) of the treatment area. Material 60 can be deposited in submucosal tissue of the intestine (e.g. in submucosa via a depositing device 600 comprising one or more needles and/or fluid jets). Step 170 can be performed, comprising procedure C. Procedure C can include one, two, or more of: restricted food intake for a minimum time period (e.g. an elemental diet for approximately 48 hours); a pureed diet for a minimum time period (e.g. at least 2 weeks); and/or intake of one or more NSAIDs.

In some embodiments, system 10 and the methods of the present inventive concepts are configured to treat a medical condition by performing a transgenic modification in the creation of material 60. For example, referring to Table 1 herebelow, a patient can be selected that has a medical condition as listed in the “Patient” column of Table 1. For each of these conditions: the patient can be screened for the parameter and level listed in the “Screen” column; a transgenic modification of tissue to create material 60 can be performed, such as is listed in the “Transgene” column; a tissue modification can be performed, such as in the location listed in the “Tissue Modification Column” (e.g. a tissue modification of mucosa or other tissue that is performed prior to depositing of material 60 and/or after depositing of material 60); and/or an adjunct therapy and/or other procedure can be performed as listed in the “Adjunct” column.

TABLE 1

| | Clinical | | Pre/peri/post | Adjunct |
|---|---|---|---|---|
| Patient | test/biomarker | Transgene | treatment | Treatment |
| Hereditary | Ferritin > 100 | Wildtype HFE | Ablation and/or | Low iron diets; |
| Hemochromatosis | | | other Tissue | phlebotomy |
| | | | Modification of | |
| | | | Duodenal | |
| | | | Mucosa | |
| Obese with Type 2 | HbA1C > 6% | GLP-1 | Ablation and/or | Delivery of |
| diabetes | Non-Asian - BMI > | GLP-1 plus PYY | other Tissue | Prebiotic |
| | 30 kg/m² | GLP-1 plus | Modification of | and/or |
| | Asian descent - | Glicentin | Duodenal | probiotic |
| | BMI > 25 kg/m² | GLP-1 plus | Mucosa | Protection (e.g. |
| | | Oxyntomodulin | | Gel protection) |
| | | GLP-1 plus CCK | | of the |
| | | | | deposited |
| | | | | Material 60 |
| Obese and Non- | HbA1C < 6% | GLP-1 | Ablation and/or | Delivery of |
| diabetic | Non-Asian - BMI > | GLP-1 plus PYY | other Tissue | Prebiotic |
| | 30 kg/m² | GLP-1 plus | Modification of | and/or |
| | Asian descent - | Glicentin | Duodenal | probiotic |
| | BMI > 25 kg/m² | GLP-1 plus | Mucosa | Protection (e.g. |
| | | Oxyntomodulin | | Gel protection) |
| | | GLP-1 plus CCK | | of the |
| | | | | deposited |
| | | | | Material 60 |
| Obese pre-T2 | HbA1c < 7%, | GLP-1 | Ablation and/or | Delivery of |
| | fasting plasma | GLP-1 plus PYY | other Tissue | Prebiotic |
| | glucose 80-122 | GLP-1 plus | Modification of | and/or |
| | mg/dl | Glicentin | Duodenal | probiotic |
| | Non-Asian - BMI > | GLP-1 plus | Mucosa | Protection (e.g. |
| | 30 kg/m² | Oxyntomodulin | | Gel protection) |
| | Asian descent - | GLP-1 plus CCK | | of the |
| | BMI > 30 kg/m² | | | deposited |
| | | | | Material 60 |
| Type 1 diabetes | Residual beta cell | GLP-1 | Ablation and/or | Delivery of |
| | function - fasting | GLP-1 plus GIP | other Tissue | Prebiotic |
| | c-peptide > 1.0 | | Modification of | and/or |
| | nM; peak c- | | Duodenal | probiotic |
| | peptide > 0.25 nM | | Mucosa | Protection (e.g. |
| | | | | Gel protection) |
| | | | | of the |
| | | | | deposited |
| | | | | Material 60 |
| MODY (maturity onset | Genetic | GLP-1 | Ablation and/or | Delivery of |
| diabetes of young), aka | screening for | GLP-1 plus GIP | other Tissue | Prebiotic |
| monogenic diabetes; | mutation p. | | Modification of | and/or |
| MODY-3 most likely | Arg200Trp in the | | Duodenal | probiotic |
| responsive | HNF1A gene | | Mucosa | Protection (e.g. |
| | (MODY-3) | | | Gel protection) |
| | | | | of the |
| | | | | deposited |
| | | | | Material 60 |
| Post-bariatric | Post-prandial | Ex 9-39 | Ablation and/or | Delivery of |
| hypoglycemia | hypoglycemia < | | other Tissue | Prebiotic |
| | 50, <70 mg/dl | | Modification of | and/or |
| | | | Duodenal | probiotic |
| | | | Mucosa | Protection (e.g. |
| | | | | Gel protection) |
| | | | | of the |
| | | | | deposited |
| | | | | Material 60 |
| Cachexia, malnutrition, | Low body weight | Ghrelin | Ablation and/or | Delivery of |
| anorexia nervosa | BMI < 18, <20 | | other Tissue | Prebiotic |
| | kg/m² | | Modification of | and/or |
| | | | Duodenal | probiotic |
| | | | Mucosa and/or | Protection (e.g. |
| | | | Gastric Fundus | Gel protection) |
| | | | | of the |
| | | | | deposited |
| | | | | Material 60 |
| Gastroparesis | Slowed gastric | Ghrelin | Ablation and/or | Delivery of |
| | emptying | | other Tissue | Prebiotic |
| | confirmed by | | Modification of | and/or |
| | radionuclide, | | Duodenal | probiotic |
| | barium meal | | Mucosa and/or | Protection (e.g. |
| | testing | | Gastric Fundus | Gel protection) |
| | | | | of the |
| | | | | deposited |
| | | | | Material 60 |
| Gastric ulcer disease, | Unresponsive to | CCK antagonism | Ablation and/or | Delivery of |
| Zollinger-Ellison | standard of care, | (CCK_{A} or CCK1) | other Tissue | Prebiotic |
| syndrome | confirmed gastric | | Modification of | and/or |
| | ulcer disease | | Duodenal | probiotic |
| | | | Mucosa | Protection (e.g. |
| | | | | Gel protection) |
| | | | | of the |
| | | | | deposited |
| | | | | Material 60 |
| Pain, anxiety, panic | Unresponsive to | CCK antagonism | Ablation and/or | Delivery of |
| attacks | standard of care | (CCK_{B} or CCK₂) | other Tissue | Prebiotic |
| | | | Modification of | and/or |
| | | | Duodenal | probiotic |
| | | | Mucosa | Protection (e.g. |
| | | | | Gel protection) |
| | | | | of the |
| | | | | deposited |
| | | | | Material 60 |
| Obese T2 | HbA1C > 6% | FGF21 (best | Ablation and/or | Delivery of |
| | Non-Asian - BMI > | candidate) | other Tissue | Prebiotic |
| | 30 kg/m² | FGF1, FGF19 | Modification of | and/or |
| | Asian descent - | (less likely | Duodenal | probiotic |
| | BMI > 25 kg/m² | applicable) | Mucosa | Protection (e.g. |
| | | | | Gel protection) |
| | | | | of the |
| | | | | deposited |
| | | | | Material 60 |
| Hypertriglyceridemia | Triglycerides > | FGF21 (best | Ablation and/or | Delivery of |
| | 150, >200 | candidate) | other Tissue | Prebiotic |
| | mg/dl | FGF1, FGF19 | Modification of | and/or |
| | | (less likely | Duodenal | probiotic |
| | | applicable) | Mucosa | Protection (e.g. |
| | | | | Gel protection) |
| | | | | of the |
| | | | | deposited |
| | | | | Material 60 |
| Obese T2 with | HbA1C > 6% | FGF21 plus GLP- | Ablation and/or | Delivery of |
| Hypertriglyceridemia | Non-Asian - BMI > | 1 | other Tissue | Prebiotic |
| | 30 kg/m² | | Modification of | and/or |
| | Asian descent - | | Duodenal | probiotic |
| | BMI > 25 kg/m² | | Mucosa | Protection (e.g. |
| | Triglycerides > | | | Gel protection) |
| | 150, >200 | | | of the |
| | mg/dl | | | deposited |
| | | | | Material 60 |
| Obese T2 with | HbA1C > 6% | FGF21 plus GLP- | Ablation and/or | Delivery of |
| NAFLD-NASH | Non-Asian - BMI > | 1 | other Tissue | Prebiotic |
| | 30 kg/m² | | Modification of | and/or |
| | Asian descent - | | Duodenal | probiotic |
| | BMI > 25 kg/m² | | Mucosa | Protection (e.g. |
| | PDFF IHTG > | | | Gel protection) |
| | 5%, elevated | | | of the |
| | ALT, elevated | | | deposited |
| | fib-4 score > 1.3 | | | Material 60 |
| Familial partial | HbA1C > 6% | GLP-1 | Ablation and/or | Delivery of |
| lipodystrophy (Type 2 | Triglycerides > | FGF21 plus GLP- | other Tissue | Prebiotic |
| Dunnigan | 200 mg/dl | 1 | Modification of | and/or |
| lipodystrophy) | Dunnigan | | Duodenal | probiotic |
| | mutation - | | Mucosa | Protection (e.g. |
| | heterozygous | | | Gel protection) |
| | R482W missense | | | of the |
| | mutations in | | | deposited |
| | protein lamin | | | Material 60 |
| | A/C (LMNA; | | | |
| | 150330) gene | | | |
| Cholestatic liver | Abnormal liver | FGF19 | Ablation and/or | Delivery of |
| disease: primary | function | | other Tissue | Prebiotic |
| biliary cirrhosis, | | | Modification of | and/or |
| primary sclerosing | | | Duodenal | probiotic |
| cholangitis, post-liver | | | Mucosa | Protection (e.g. |
| transplantation | | | | Gel protection) |
| | | | | of the |
| | | | | deposited |
| | | | | Material 60 |
| Post-ileal resection | >4 stools/day | FGF19 | Ablation and/or | Delivery of |
| biliary diarrhea | | | other Tissue | Prebiotic |
| | | | Modification of | and/or |
| | | | Duodenal | probiotic |
| | | | Mucosa | Protection (e.g. |
| | | | | Gel protection) |
| | | | | of the |
| | | | | deposited |
| | | | | Material 60 |

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. (canceled)

2. A method of treating a medical condition of a patient, comprising:
selecting a patient;
harvesting tissue from a harvest site a mammalian subject via a harvesting device; and
depositing material at a deposit site in the patient via a depositing device, the material being based on the harvested tissue;
wherein the deposited material is configured to generate resultant tissue configured to treat the medical condition of the patient,
wherein the medical condition treated comprises Type 2 diabetes.

3. The method according to claim 2, wherein the medical condition treated further comprises a medical condition selected from the group consisting of: Type 1 diabetes; double diabetes; gestational diabetes; hyperglycemia; pre-diabetes; monogenic diabetes; maturity onset diabetes of the young; impaired glucose tolerance; insulin resistance; hyperinsulinemia; hypoinsulinemia; non-diabetic hypoglycemia; non-alcoholic fatty liver disease; non-alcoholic steatohepatitis; obesity; obesity-related disorder; polycystic ovarian syndrome; hypertriglyceridemia; hypercholesterolemia; psoriasis; Alzheimer's disease; and combinations thereof.

4. The method according to claim 2, wherein the harvest site comprises an anatomical location selected from the group consisting of: the gastrointestinal tract; the mouth; the esophagus; the stomach; the duodenum; the jejunum; the ileum; the colon; an organ; the brain; the lungs; the liver; the bladder; the kidneys; the heart; the small intestine; the large intestine; the skin; the peritoneal cavity; and combinations thereof.

5. The method according to claim 2, wherein the harvest site comprises one or more locations in the ileum and/or colon.

6. The method according to claim 2, wherein the harvested tissue comprises tissue selected from the group consisting of: mucosal tissue; submucosal tissue; tissue comprising at least one stem cell; tissue comprising at least one stem cell of the mucosa; tissue comprising at least one mucosal crypt containing a stem cell; mucosal tissue comprising at least one stem-cell containing crypt; organoids; epithelial layer tissue; uroepithelial layer tissue; intestinal epithelial layer tissue; lung epithelial layer tissue; and combinations thereof.

7. The method according to claim 2, wherein the deposit site comprises an anatomical location selected from the group consisting of: the gastrointestinal tract; the mouth; the esophagus; the stomach; the duodenum; the jejunum; the ileum; the colon; an organ; the brain; the lungs; the liver; the bladder; the kidneys; the heart; the small intestine; the large intestine; the skin; the peritoneal cavity; and combinations thereof.

8. The method according to claim 2, wherein the deposit site comprises one or more locations in the duodenum and/or proximal jejunum.

9. The method according to claim 2, wherein the deposit site comprises an anatomical location selected from the group consisting of: luminal wall tissue of the gastrointestinal tract; mucosal tissue of the gastrointestinal tract; submucosal tissue of the gastrointestinal tract; the peritoneal cavity; and combinations thereof.

10. The method according to claim 2, further comprising processing the harvested tissue prior to deposition in the patient.

11. The method according to claim 10, wherein the harvested tissue is amplified.

12. The method according to claim 10, wherein the harvested tissue is transgenically modified.

13. The method according to claim 2, wherein the depositing of the material is configured to modify neurohormonal signaling in the gastrointestinal tract.

14. The method according to claim 2, wherein the depositing device comprises at least two depositing elements configured to deposit the material at the deposit site.

15. The method according to claim 2, wherein the material further comprises a protecting element configured to protect the material after depositing of the material at the deposit site.

16. The method according to claim 2, further comprising treating tissue proximate the deposit site via a treatment device prior to depositing the material.

17. The method according to claim 16, wherein the treatment device comprises a tissue ablation device configured to ablate tissue proximate the deposit site.

18. The method according to claim 17, wherein the tissue ablation device is configured to ablate mucosal tissue proximate the deposit site.

19. The method according to claim 16, wherein the treatment device comprises a tissue expansion device configured to expand tissue proximate the deposit site.

20. The method according to claim 2, wherein the patient is selected to have a minimum level of pancreatic functionality.

21. The method according to claim 20, wherein the minimum level of pancreatic functionality is based on the patient's c-peptide level.
